(19) United States
(12) Patent Application Publication
(10) Pub. No.: US 2025/0231299 A1
(43) Pub. Date: Jul. 17, 2025

(54) System and Method/Process for In-Field Measurements of Plant Crops

(71) Applicant: Agriculture Victoria Services Pty Ltd, Bundoora, Victoria (AU)

(72) Inventors: German Carlos Spangenberg, Bundoora, Victoria (AU); Bikram Pratap Banerjee, Bundoora, Victoria (AU); Surya Kant, Bundoora, Victoria (AU)

(21) Appl. No.: 18/700,474

(22) PCT Filed: Oct. 11, 2022
(86) PCT No.: PCT/AU2022/051218

§ 371 Date: Apr. 11, 2024

(30) Foreign Application Priority Data
Oct. 12, 2021 (AU) 2021903273

(51) Int. Cl. G01S 17/89; G01B 11/06; G01N 21/84; G01S 7/00; G01S 7/48; G01S 7/481

(52) U.S. Cl. CPC G01S 17/89; G01B 11/0608; G01N 21/84; G01S 7/003; G01S 7/4808; G01N 2021/8466; G01N 2201/0216; G01S 7/4813

(57) ABSTRACT

A measurement system including: a. a sensor system that includes: i. a Light Detection And Ranging (LiDAR) module with a laser emitter configured to generate measurement data representing raw range measurements to measure heights of a crop, and ii. a computing module, including: at least one wireline/wired communications module configured to communicate with the LiDAR module for the computing module to acquire the measurement data from the LiDAR module; and at least one wireless communications module configured for the computing module to communicate using a wireless connection/link with a remote computing system that is configured receive the acquired measurement data and to determine/calculate/estimate phenotypic quantities of the crop based on the measured heights for the purpose of high-throughput plant phenotyping (HTPP); and b. a mobile/vehicle mount configured to hold/support the sensor system above the crop and to direct the laser emitter towards the crop.

### RELATED APPLICATION

The present patent application is related to Australian Provisional Patent Application No. 2021903273, filed 12 Oct. 2021 in the name of Agriculture Victoria Services Pty Ltd and entitled “System and method/process for in-field measurements of plant crops”, the originally filed specification of which is hereby incorporated by reference in its entirety.

### TECHNICAL FIELD

The present disclosure relates to a system and a method/process for non-destructive in-field measurements of plant crops, e.g., for in-field crop phenotype measurements/estimations (e.g., height and biomass/biovolume), e.g., including for high-throughput plant phenotyping (HTPP) and remote/non-contact sensing/measurements.

### BACKGROUND

Phenotypic characterization of crop genotypes is an essential yet challenging aspect of crop management and breeding research. Crop biomass and height may be fundamental morphological traits to estimate crop growth and selection of genotypes of interest in a breeding program. Crop biomass is associated with plant growth and development, being the basis of vigour and net primary productivity. Crop biomass is a measure of the total fresh weight (FW) or dry weight (DW) of organic matter per unit area, which are measured by destructively harvesting plants and weighing for FW, and oven drying and weighing to get DW. Plant height is the vertical distance from ground level to the upper boundary of the primary photosynthetic tissues, and conventionally measured in field using rulers. These manual and destructive data collection methods are highly inefficient, laborious, operationally expensive and prone to manual error. Applicability of manual methods are limited to small field experimental trials and are not scalable and repeatable for large field experimental trials.

Digital sensing technologies are rapidly advancing plant phenotyping and speeding-up breeding outcomes. However, existing sensors might not be fully applicable and suitable for agriculture research due to diversity in crop species and specific need during selection of preferred genotypes. Furthermore, existing digital sensor units may be too large, heavy and/or expensive for some applications, e.g., HTPP, and/or may require post-processing of too much data (e.g., a large number of images to accurately construct depth models, or large data output from 360-degree Light Detection And Ranging devices (LiDARs)), thus requiring enormous computational power.

It is desired to address or ameliorate one or more disadvantages or limitations associated with the prior art, or to at least provide a useful alternative.

### SUMMARY

In accordance with the present invention, there is provided a measurement system 100 including:
a. a sensor system 300 that includes:
   i. a Light Detection And Ranging (LiDAR) module 302 with a laser emitter 318 configured to generate measurement data representing raw range measurements to measure heights of a crop 104 (of plants, e.g., a plot or field with abutting plants in both horizontal dimensions, including pasture crops), and
   ii. a computing module 304, including:
      at least one wireline/wired communications module configured to communicate with the LiDAR module 302 for the computing module 304 to acquire the measurement data (e.g., including a USB module with a USB port, and/or a general-purpose input/output module with GPIO port) from the LiDAR module 302; and
      at least one wireless communications module 326 (e.g., a wireless local area network (WLAN) module, a cellular module, and/or a cellular Internet-of-Things (IoT) module) configured for the computing module 304 to communicate using a wireless connection/link 110 with a remote computing system 106 (e.g., which can include a cloud-computing server access via the Internet 108) that is configured receive the acquired measurement data and to determine/calculate/estimate phenotypic quantities of the crop 104 based on (processed data representing) the measured heights for the purpose of high-throughput plant phenotyping (HTPP); and
b. a mobile/vehicle mount 102 configured to hold/support the sensor system 300 above the crop 104 and to direct the laser emitter 318 towards the crop 104.

The sensor system 300 may include at least one sensor case 500 that is configured to surround, enclose and encase electronic circuitry portions of the LiDAR module 302 and the computing module 304 to seal off the enclosed circuitry portions to mitigate/stop ingress of moisture/dust/dirt while the sensor system 300 is operating in a field. The sensor case 500 may include a plurality of portions (or parts/pieces/sides) formed/manufactured of an additive/3D printing material (using an additive/3D printer). The plurality of portions may be mutually assembled/fastened by threaded fasteners. The sensor case 500 may include compressible/deformable seals/gaskets between mutually assembled ones of the portions, optionally wherein the mobile/vehicle mount 102 is configured to hold/support the power case/housing such that laser emitter is directed towards the crop 104.

The sensor system 300 may include a power source 310. The power source 310 may include a battery 312 that powers the LiDAR module 302. The power source 310 may include a DC-to-DC converter 314, powered by the battery 312, that provides a different voltage from that powering the LiDAR module 302 to power the computing module 304. The power source 312 may include a power case/housing that surrounds, encloses and encases electronic circuitry portions of the power source 310 to seal off the enclosed circuitry portions to mitigate/stop ingress of moisture/dust/dirt while the power source 310 is operating in a field. The mobile/vehicle mount 102 may be configured to hold/support the power case/housing such that the power source 310 is electrically connected/connectable to the LiDAR module 302 and the computing module 304.

The sensor system 300 may include a global navigation satellite system (GNSS) module 306 with a GNSS receiver 308 configured to simultaneously measure the geolocation of the sensor system 300 while the LiDAR module 302 is measuring the heights. The computing module 304 may include at last one wireline/wired communications module configured to communicate with the GNSS module 306 for the computing module 304 to receive the geolocation data (e.g., including a USB module with a USB port, and/or a general-purpose input/output module with GPIO port). The sensor case 500 may be configure to surround, enclose and encase electronic circuitry portions of the GNSS module 306 to seal off the enclosed circuitry portions to mitigate/stop ingress of moisture/dust/dirt while the sensor system 300 is operating in a field.

The sensor system 300 with the LiDAR module 302, the computing module 304, the GNSS module 306 (with the GNSS receiver 308) and the sensor case 500 may have a weight of less than 1 kilogram (kg), or less than 550 grams (g), or between 350 and 500 g. The LiDAR module 302 may have a weight of less than 200 g, the computing module 304 may have a weight of less than 50 g, the GNSS module 306 (with the GNSS receiver 308) may have a weight of less than 100 g, and/or the sensor case 500 may have a weight of less than 200 g.

The LiDAR module 302 may include a LiDAR sensor 303 configured for one-dimensional scanning (which is side-to-side scanning or “across-track” scanning when in use), optionally in a horizontal across-track scanning direction that is at least partially, and typically substantially, perpendicular to a horizontal along-track travel direction of the mobile/vehicle mount 102 (the “track” is the travel direction or route of the mobile/vehicle mount 102). The 1D scanning LiDAR sensor 303 (i.e., configured for 1D scanning) may include a solid-state LiDAR sensor. The solid-state LiDAR sensor may include a micro-electromechanical system (MEMS) chip or an optical phased array. The solid-state LiDAR sensor is configured to steer a laser beam from the laser emitter 318 along the horizontal scanning direction (side-to-side or across-track when in use). By steering the laser beam using solid-state components of the LiDAR sensor, which can be the MEMS chip or phased array, the solid-state LiDAR sensor may have no mechanical moving parts larger than elements of a MEMS chip, e.g., no mechanical moving parts with an average diameter larger than 0.1 mm. The 1D scanning may be over the horizontal across-track scanning distance that corresponds to a side-to-side or across-track field of view (FoV) of the LiDAR sensor, optionally wherein the across-track FoV is less than 90 degrees, or less than 60 degrees, optionally wherein the LiDAR sensor has a front-to-back or along-track FoV that is substantially perpendicular to the across-track FoV and that is substantially less than the across-track FoV, optionally wherein the along-track FoV is less than 1 degree or substantially 0.3 degrees. By scanning the laser beam over a limited horizontal scanning distance, corresponding to a FoV less than 90 or 60 degrees, the data output from the LiDAR module 302 may be substantially less than if a larger distance or area were scanned.

The computing module 304 (in its onboard memory) may include credentials (including a password and/or a subscriber identity module (SIM)) configured to automatically connect to a wireless network 112 via the wireless connection/link 110. The wireless connection/link 110 may include a radio-frequency carrier.

The mount 102 may include a ground vehicle/mount with wheels configured to roll the sensor system 300 along ground/soil under the crop in a travel direction of the mount 102 that is at least partially transverse to a horizontal scanning direction of the laser emitter 318, optionally wherein the mount 102 is configured to hold/support the LiDAR module 302 at a selected height above the ground/soil while the LiDAR module 302 is measuring the heights.

The measurement system 100 may include the remote computing system 106. The remote computing system 106 may include machine-readable memory (“server memory”) and one or more microprocessors (“server microprocessors”) connected to perform operations by executing server operational modules (“server modules”) that include data processing modules (referred to as “high-level processing nodes”) that include any one or more of:
a. a calibration module configured to determine calibrated range measurements from the raw range measurements and a stored calibration model (representing calibration measurements and true calibration distances);
b. a range-to-height conversion module configured to control the server microprocessors to convert the (raw or calibrated) range measurements (r) into crop height measurements (h);
c. a denoising module configured to mitigate spurious/noisy disturbances in the crop height measurements (h) due to undulations of ground under the crop 104 by performing a denoising process on the crop height measurements (h), including:
   i. filtering the crop height measurements (h) with a smoothing filter,
   ii. removing ground-surface heights in the crop height measurements (h) using a vertical threshold (e.g., Vₜₕ<5 cm) to remove undulating ground surface heights, and/or
   iii. removing false peaks under a horizontal threshold (Hₜₕ) lengthwise scan size of samples (e.g., Hₜₕ<50 samples);
d. a segmentation module configured to automatically segment the crop height measurements (h) into a plurality of mutually separate plot profiles corresponding to respective mutually separate plots of the crop 104 along a direction of travel of the mount 102;
e. a speed-compensation module configured to automatically compensate for variable speed of movement of the sensor system 300 along a direction of travel of the mount 102 by resampling the crop height measurements (h) to a constant selected rate for each of the plurality of separate plot profiles;
f. an edge-compensation module configured to automatically remove or add edges from/to the crop height measurements (h, H) corresponding to range measurements from outer detector elements of the LiDAR module 302 by automatically adjusting the height values of these edges;
g. optionally a geolocation module configured to automatically geolocate the crop height measurements (h, H), based on geolocation data/tags from the GNSS module 306;
h. a phenotypic module configured to automatically control the remote microprocessor to calculate/measure/estimate a phenotypic measurement from the height measurements, optionally wherein the phenotypic measurement includes a biovolume measurement, and the phenotypic module includes a biovolume module configured to automatically control the remote microprocessor to calculate/measure/estimate a crop biovolume (BV) from the height measurements;
i. a master data repository configured to store the range measurements, the crop height measurements, the phenotypic measurements, and/or geolocation data; and
j. an output module configured to automatically output the phenotypic measurements to machine-readable memory and/or to a user device for display to a user.

In accordance with the present invention, there is provided a measurement method/process 200 that includes:
a. a sensor system automatically measuring heights of a crop 104 using Light Detection And Ranging (LiDAR) while being held/supported by a mount 102 moving over/across the crop 104 (202); and
b. the sensor system automatically wirelessly sending data representing the corresponding measured heights to a remote computing system 106 (206) for high-throughput plant phenotyping (HTPP).

The measurement method/process 200 includes:
a. the remote computing system automatically determining/calculating/estimating phenotypic quantities (“phenotypic measurements”) of the crop 104 based on the received data representing the corresponding measured heights (208) for the purpose of HTPP; and
b. the remote computing system automatically outputting the phenotypic measurements (210) to machine-readable memory (e.g., the master data repository) and/or to a user device for display to a user (e.g., a farmer or scientist).

### BRIEF DESCRIPTION OF THE DRAWINGS

Some embodiments of the present invention are hereinafter described, by way of example only, with reference to the accompanying drawings, in which:

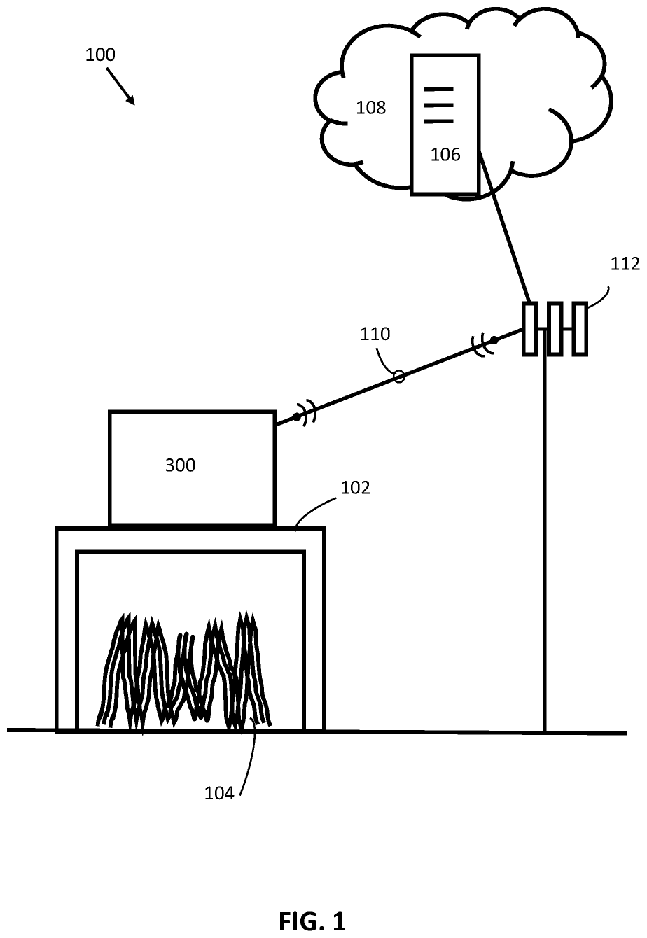

a. FIG. 1 is a schematic diagram of a system (“measurement system”) configured for making in-field measurements of field plant crops;

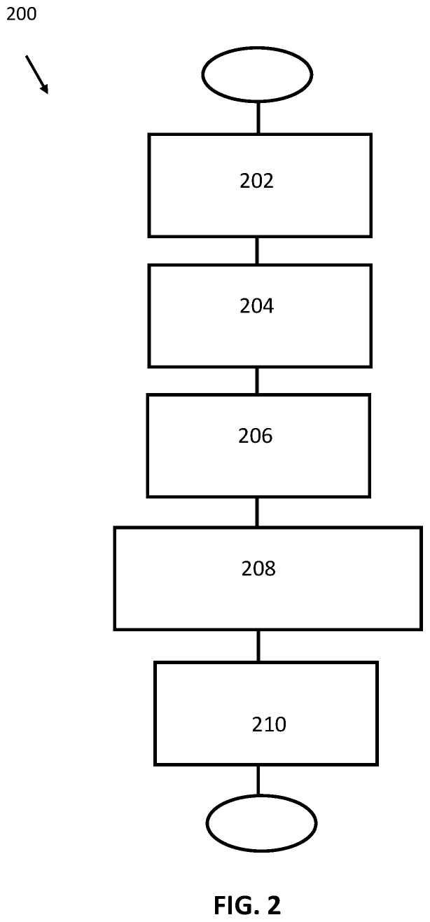

b. FIG. 2 is a flowchart of a method (“measurement method”) of making in-field measurements of field plant crops;

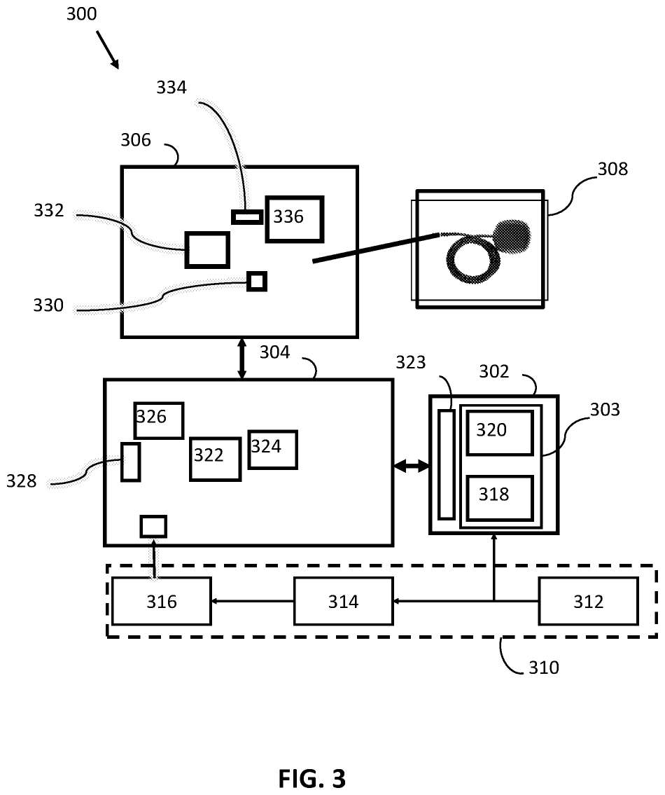

c. FIG. 3 is block diagram of a sensor system of the measurement system;

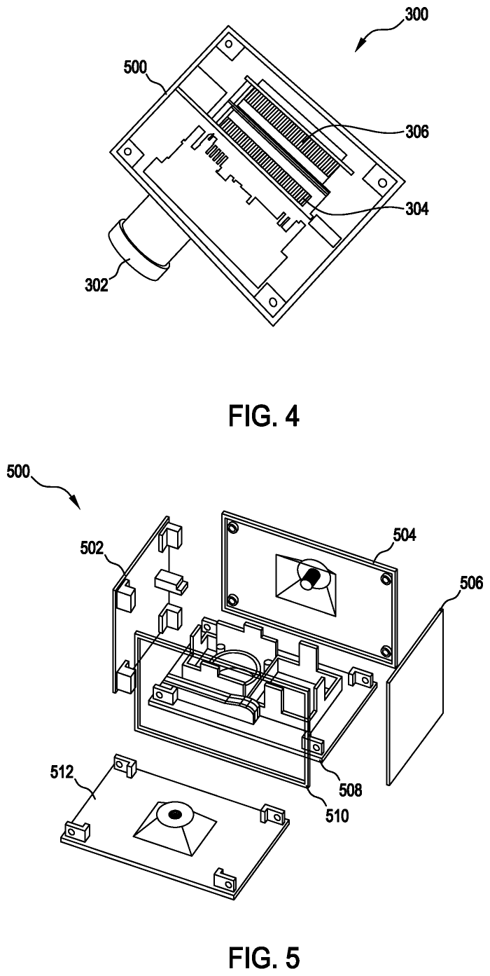

d. FIG. 4 is a photograph of electronic circuitry portions of the sensor system inside a sensor case;

e. FIG. 5 is a perspective diagram of side parts for the sensor case of the sensor system;

f. FIG. 6 is a photograph of a calibration apparatus of the measurement system;

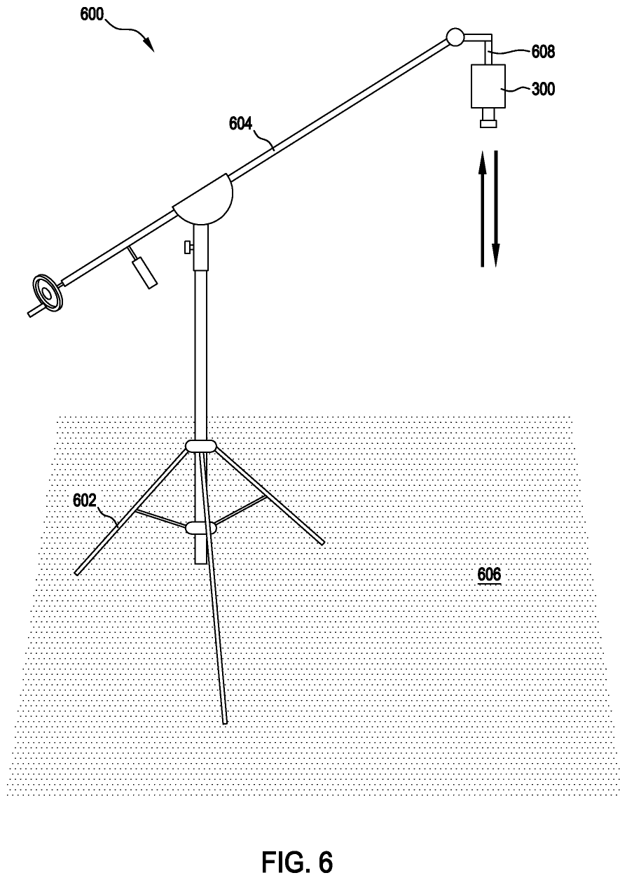

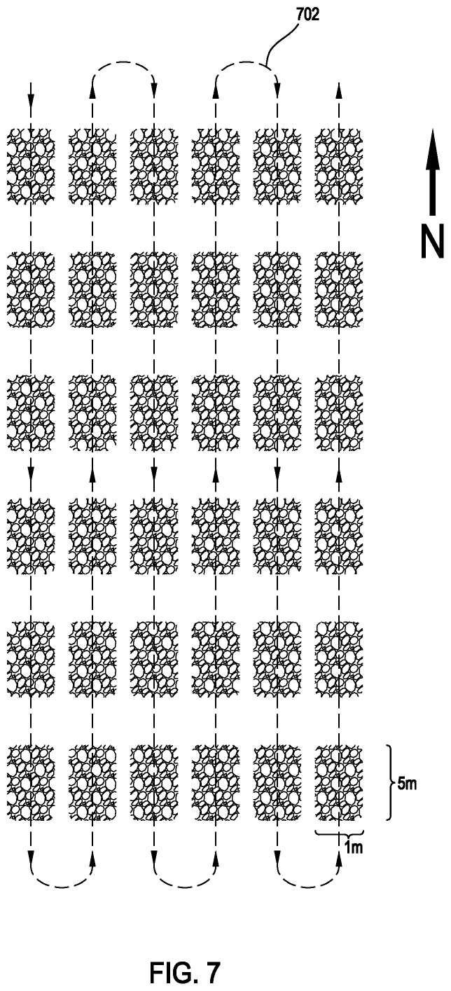

g. FIG. 7 is a diagram of a trajectory/path/pattern of the sensor system over a plurality of field plots with respective field plant crops;

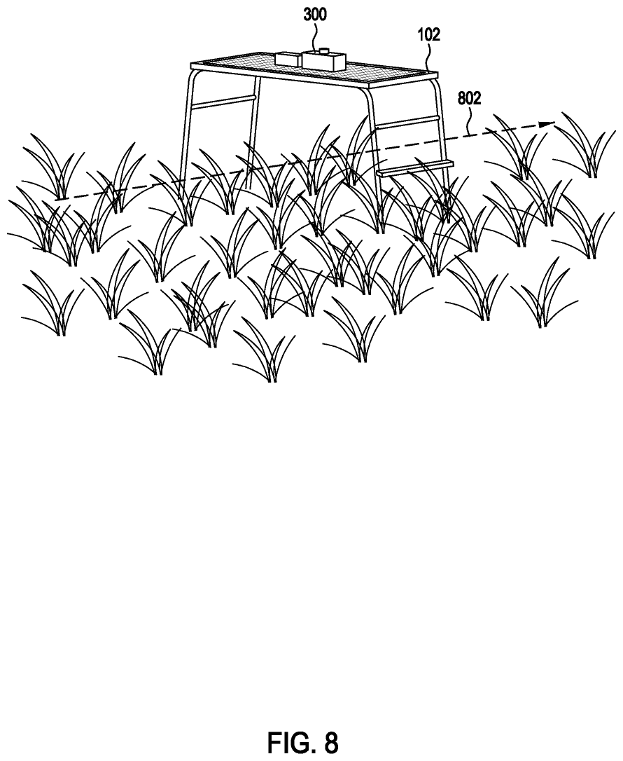

h. FIG. 8 is a perspective photograph of the sensor system on a mobile vehicle mount;

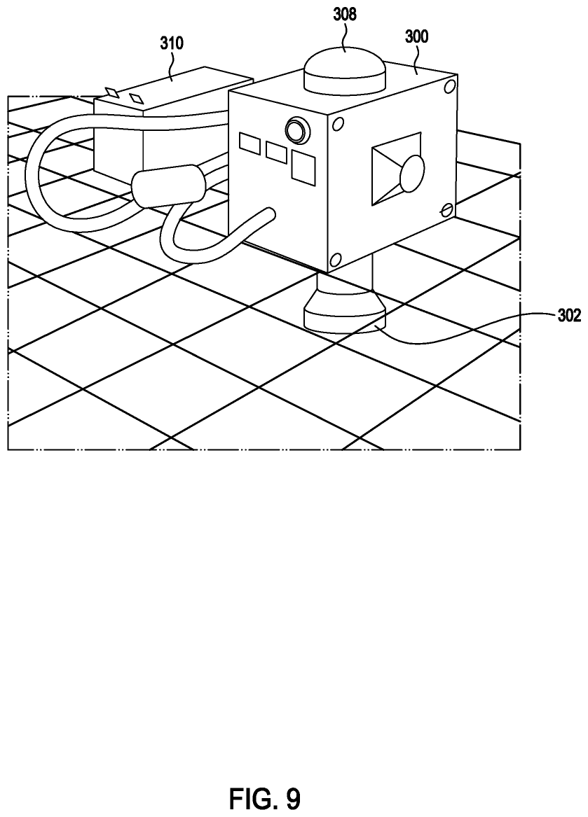

i. FIG. 9 is a perspective photograph of the sensor system showing its orientation/position on the mount;

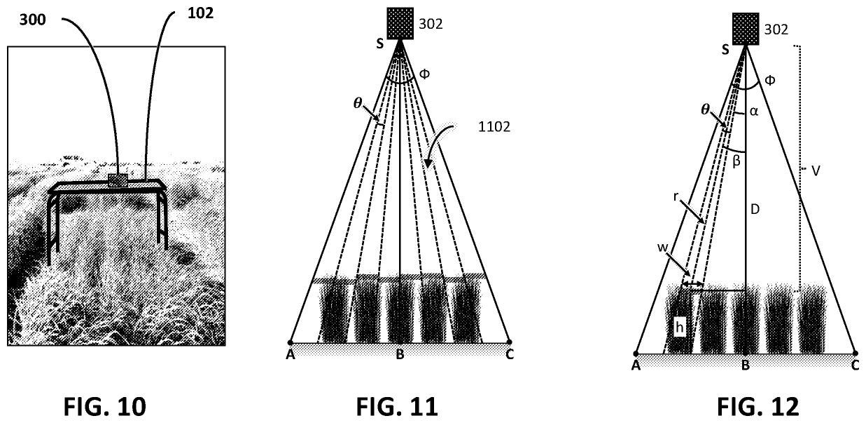

j. FIG. 10 is a front-view photograph of the sensor system on the mount;

k. FIG. 11 is a front-view diagram of LiDAR beam segments extending downwards from the sensor system to the field crop;

l. FIG. 12 is a front-view diagram of the LiDAR beam segments' geometry;

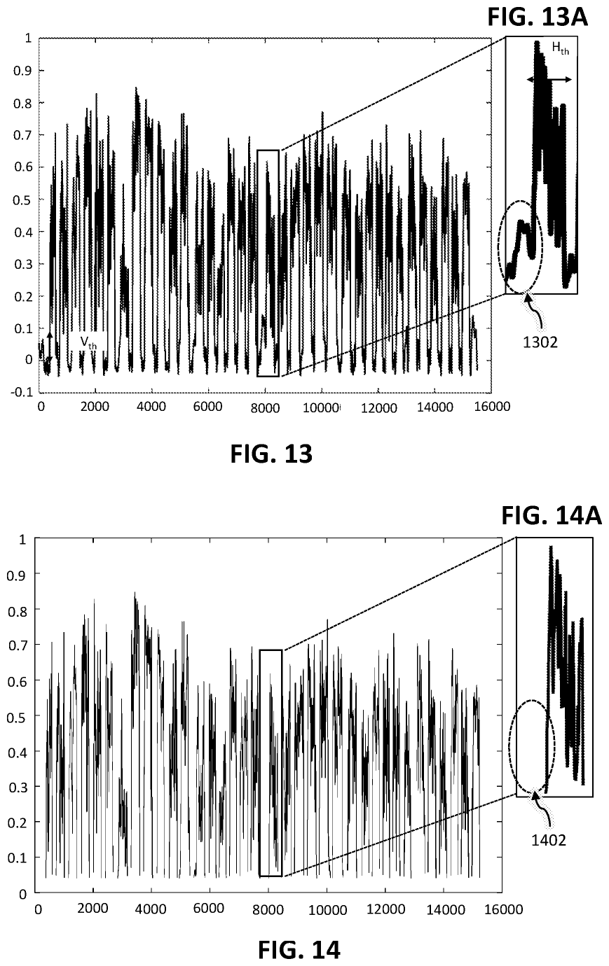

m. FIG. 13 is a graph of height profile in meters (Y axis) versus LiDAR return samples (X axis) of an example continuous height profile, measured by the system, with spurious noise and false peaks;

n. FIG. 13A is an expanded portion of the graph marked with a rectangle in FIG. 13 showing a false peak;

o. FIG. 14 is a graph of height profile in meters (Y axis) versus LiDAR return samples (X axis) of a processed example continuous height profile showing removal of noise, false peaks, and ground profile from the example continuous height profile of FIG. 13 by segmentation and classification;

p. FIG. 14A is an expanded portion of the graph marked with a rectangle in FIG. 14 showing removal of the false peak;

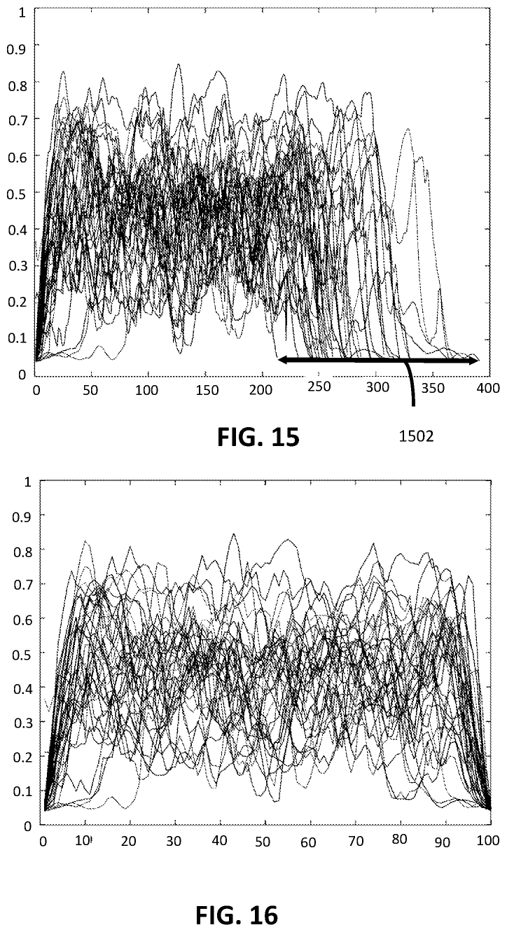

q. FIG. 15 is a graph of height profile in meters (Y axis) versus LiDAR return samples (X axis) of a plurality of classified extracted plot profiles from the continuous height profile of FIG. 14;

r. FIG. 16 is a graph of height profile in meters (Y axis) versus LiDAR return samples (X axis) of a plurality of resampled plot scans from the extracted plot profiles of FIG. 15;

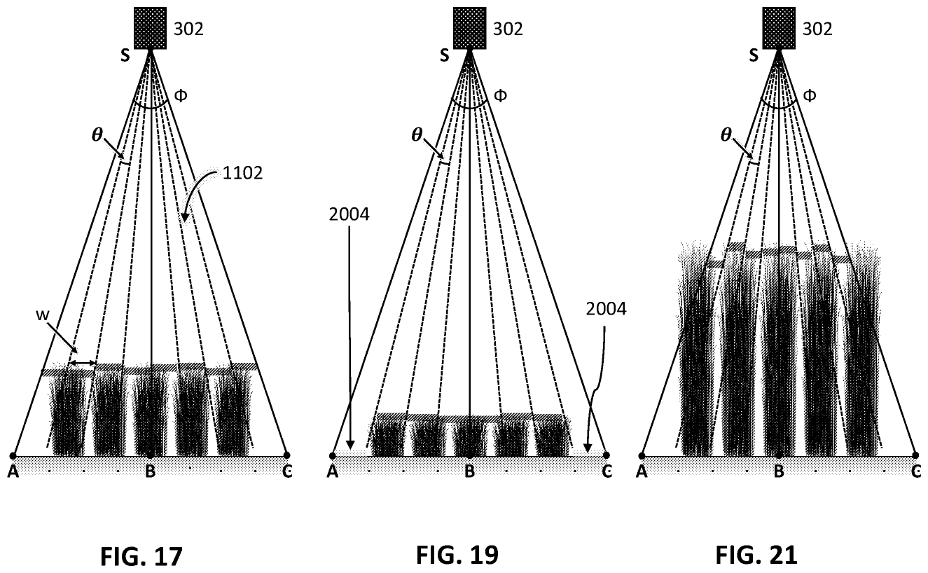

s. FIG. 17 is a front-view diagram of the LiDAR geometry for a plot of medium-sized plants;

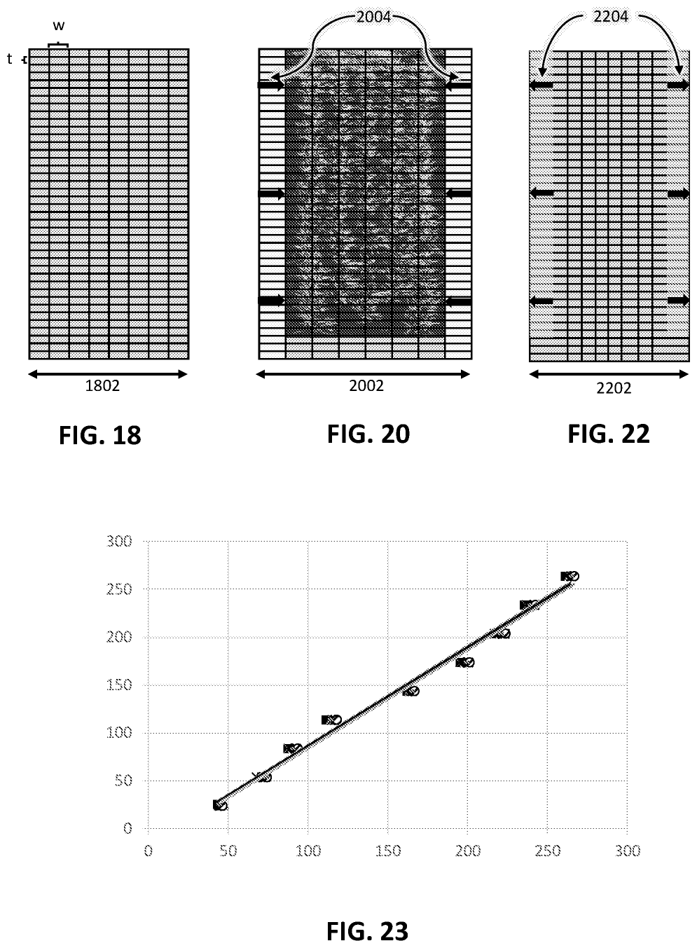

t. FIG. 18 is a top-view diagram of the LiDAR footprint on the plot of medium-sized plants with a grid of LiDAR segments (left to right) and LiDAR samples/scans (bottom to top);

u. FIG. 19 is a front-view diagram of the LiDAR beam segment geometry for a plot of small-sized plants showing where pulses return from the group on the left and right edges (edge segments);

v. FIG. 20 is a top-view diagram of the LiDAR footprint on the plot of small-sized plants with a grid of LiDAR segments (left to right) and LiDAR samples/scans (bottom to top), showing removal of the edge segments;

w. FIG. 21 is a front-view diagram of the LiDAR beam segment geometry for a plot of tall-sized plants;

x. FIG. 22 is a top-view diagram of the LiDAR footprint on the plot of tall-sized plants with a grid of LiDAR segments (left to right) and LiDAR samples/scans (bottom to top), showing extrapolation of the edge segments;

y. FIG. 23 is a graph of an example model between sensor measured distances in cm (X axis) and reference distances in cm (Y axis), with respective measurements of eight sensor elements as dots, and fitted regression models for the respective detector elements as lines;

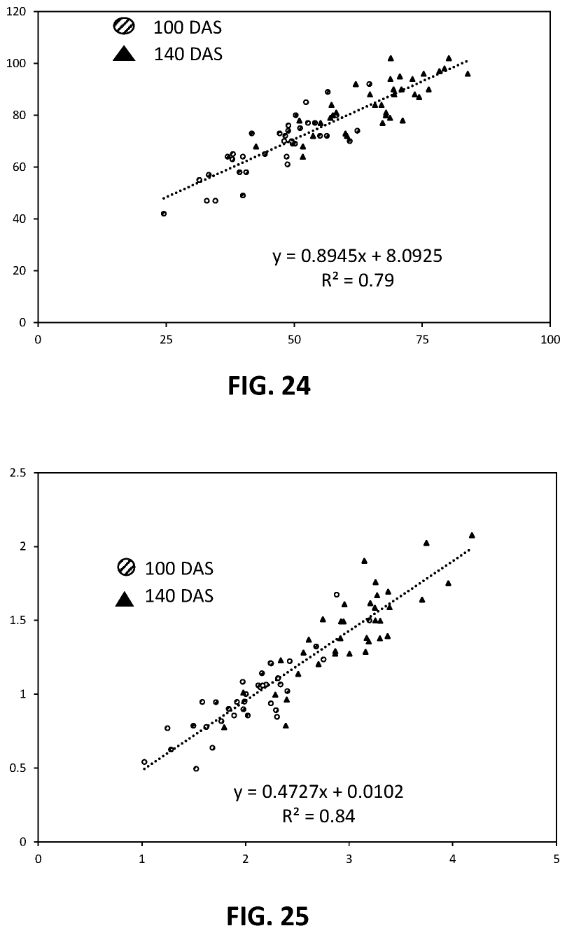

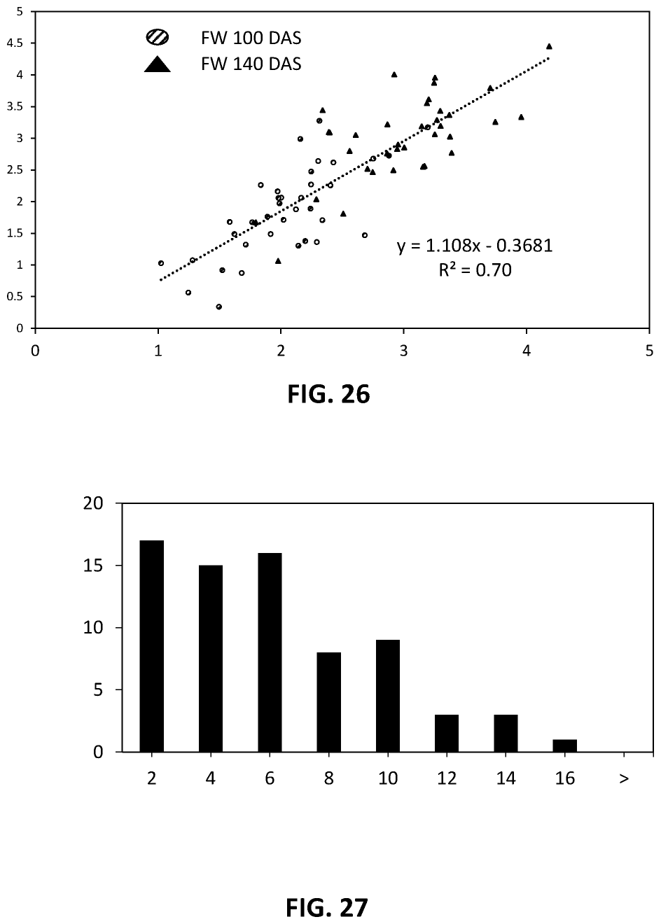

z. FIGS. 24 to 26 are graphs of example sensor measurements (X axes) and manual ground-truth (GT) measurements (Y axes) for example crops measured at 100 DAS (circles) and 140 DAS (triangles), and corresponding regression lines (dotted lines), wherein the measurements are of: GT (Y) versus sensor-measured plant height in cm (X) in FIG. 24, GT dry biomass in kg (Y) versus sensor-measured biovolume in cubic metres (X) in FIG. 25, and GT fresh biomass in kg (Y) versus sensor-measured biovolume in cubic metres (X) in FIG. 26;

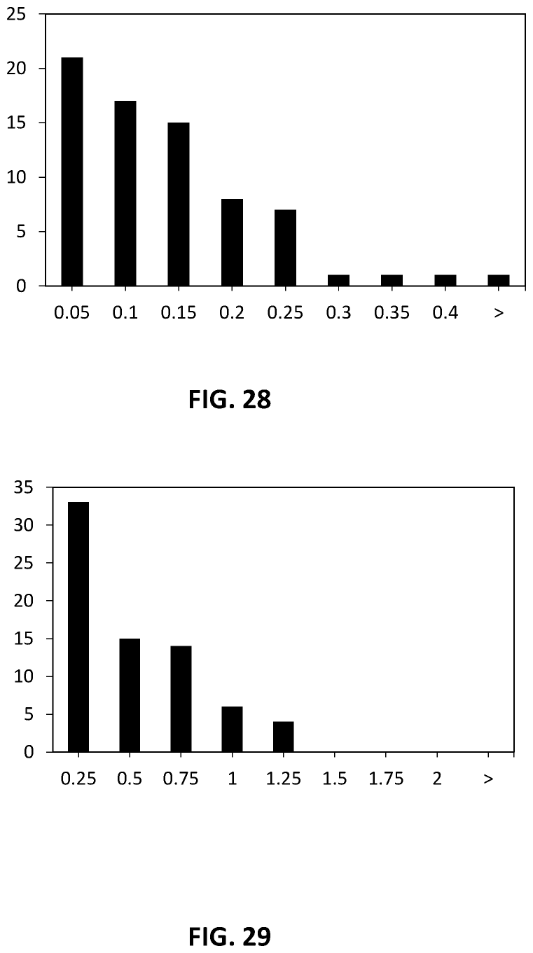

aa. FIGS. 27 to 29 are column graphs of frequencies (Y axes) of absolute error measurements (X axes) in the example sensor measurements of FIGS. 24 to 26, wherein the absolute error measurements are in the example measurements of: plant height (in cm) in FIG. 27, dry biomass (in kg) in FIG. 28, and fresh biomass (in kg) in FIG. 29;

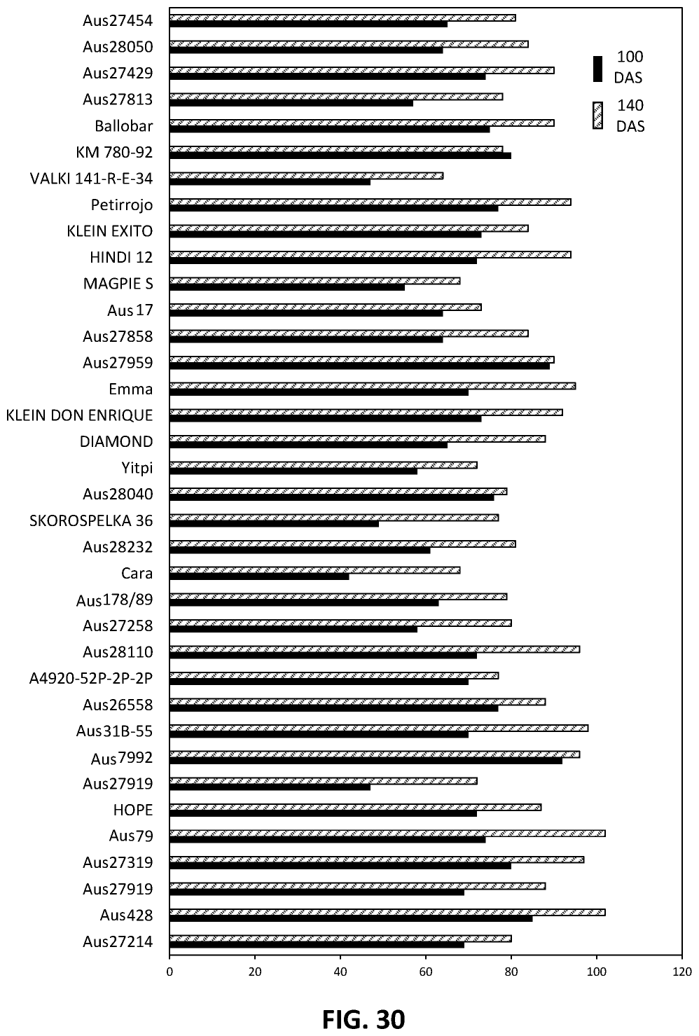

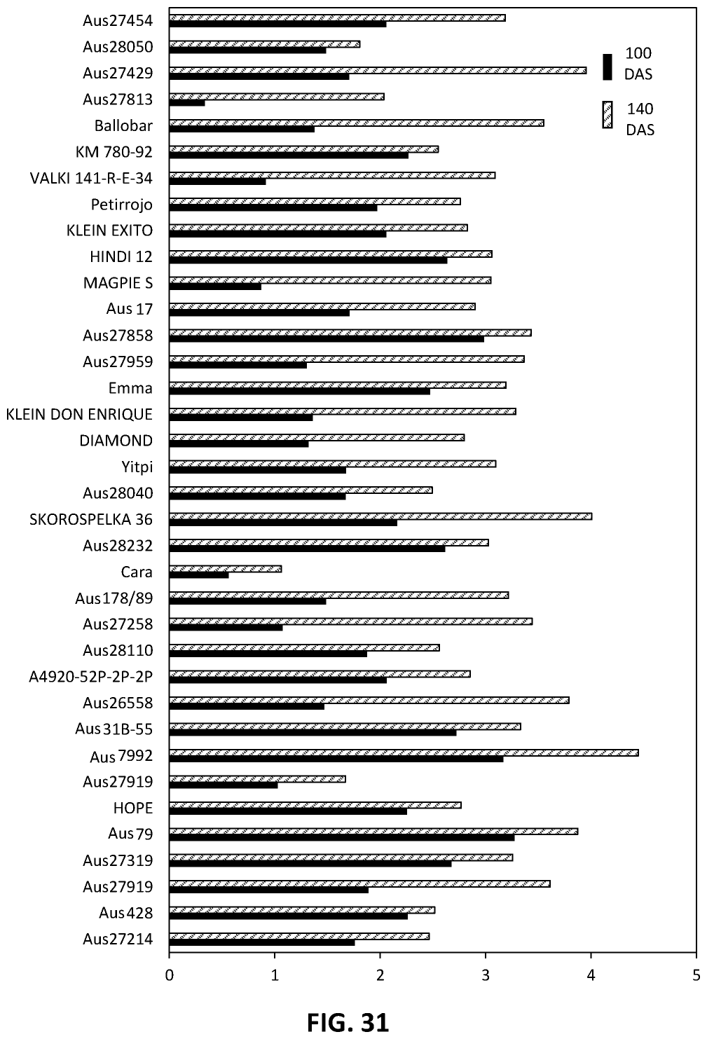

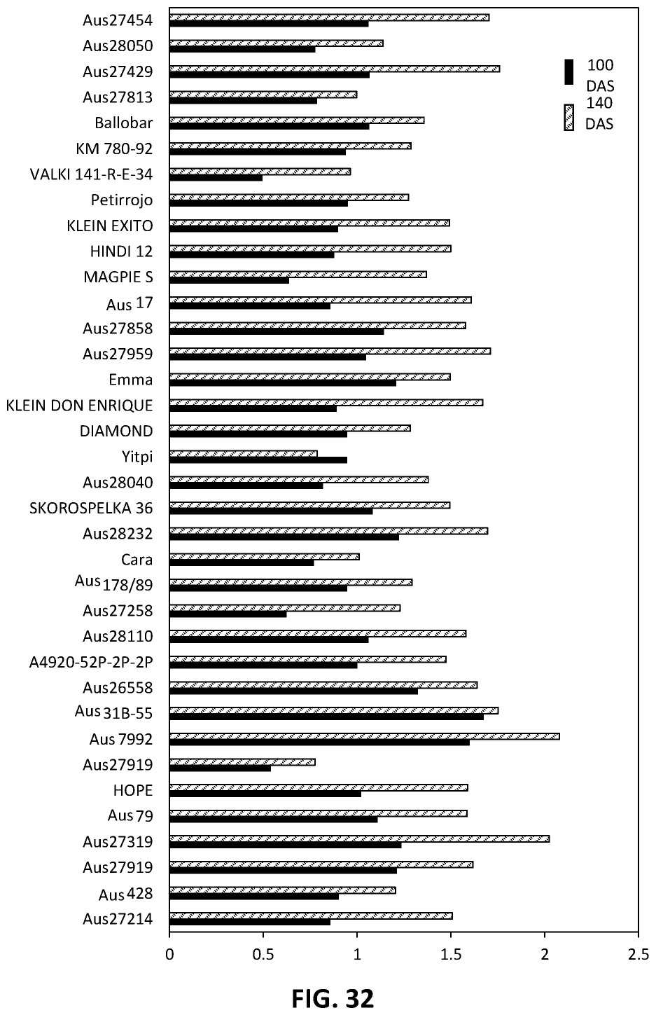

bb. FIGS. 30 to 32 are bar graphs of example phenotypic measurements (X axes) for a plurality of different wheat genotypes (Y axes), measured at 100 DAS (solid colour columns) and 140 DAS (patterned columns), wherein the phenotypic measurements are of: plant height (in cm) in FIG. 30, fresh weight/biomass (kg) in FIG. 31, and dry weight/biomass (kg) in FIG. 32; and

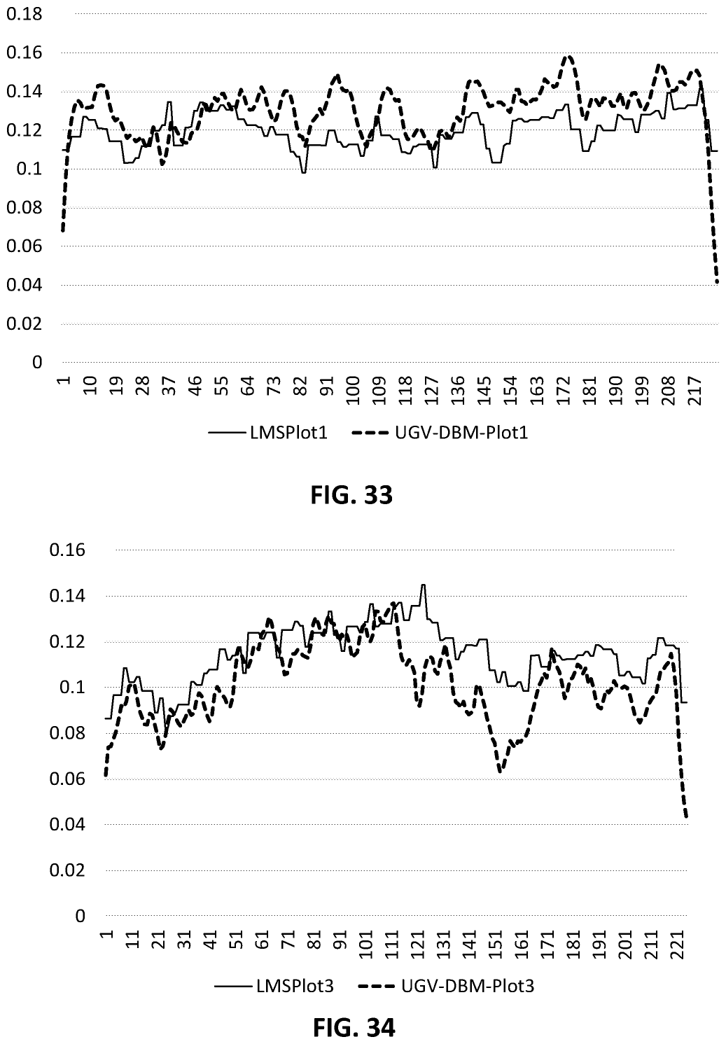

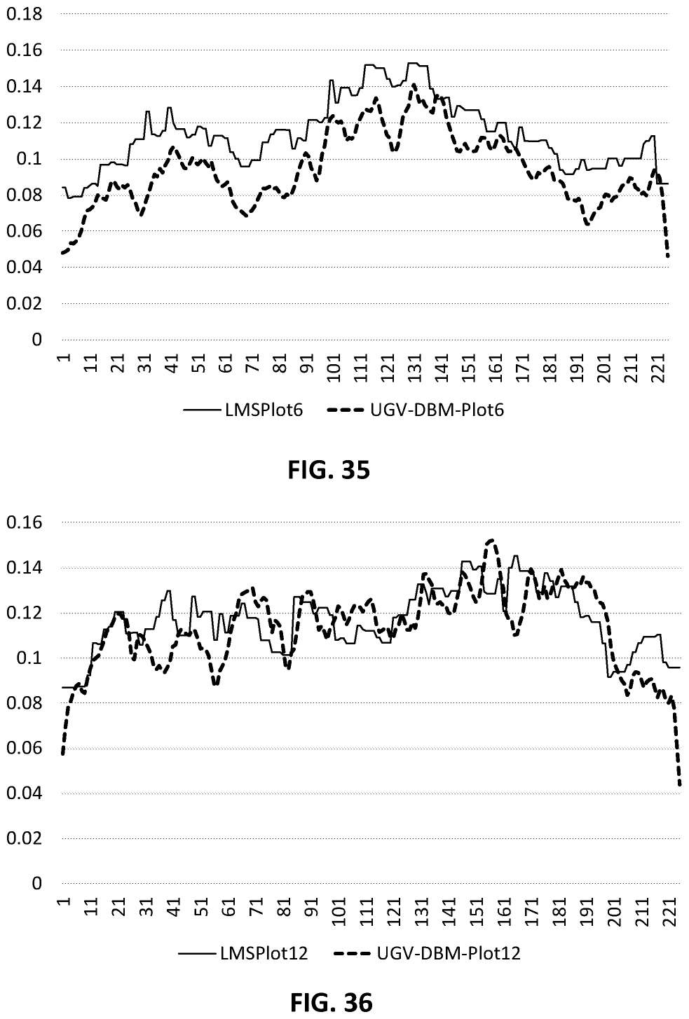

cc. FIGS. 33 to 36 are plots of measured field plant height in metres (Y axis) versus 1D distance along/across the plot in measurement bins which correlate linearly to distance (X axis) for four respective example plots of ryegrass measured in a trial using both: (a) the measurement system disclosed herein (shown in dashed lines) and (b) a commercially available 2D scanner (“LMS400”) for comparison (shown in unbroken lines).

### DETAILED DESCRIPTION

### Overview

Described herein is a measurement system, a measurement method, and a sensor system that is Internet-of-Things (IoT)-enabled by way of wireless communication with a remote computing system (which can include a cloud-computing server access via the Internet) and global navigation satellite systems (GNSS), and that uses light distance-and-ranging (LiDAR) to provide non-destructive high-throughput in-field plant phenotyping, including crop height and biomass measurements, for crop monitoring (while leaving the crop alive in the field) and management for precision agricultural applications. In particular embodiments, the plant crop is a field crop plant and/or greenhouse crop plant, particularly a cereal crop, a pasture crop, a vegetable crop, an oil-seed crop, or a Cannabis crop. The field plant crop or greenhouse crop includes many plants that are mutually closely spaced in the field or plot or greenhouse—e.g., grain-type or pasture crops such as wheat, tall fescue, barley, ryegrass, lucerne (and/or other tall cereal/pasture crops or short cereal/pasture crops), field peas and lentils (and/or other vegetable crops), oil-seed crops (such as canola, safflower, sunflower, soybeans), or Cannabis—such that the plants can be described as being in a field or pasture or greenhouse, mutually abutting in both horizontal dimensions, which is in contrast to non-field crops, e.g., orchard crops like fruit trees, that are mutually spaced, e.g., to allow people and machinery to move between mutually adjacent trees.

The sensor system may be low in weight, low in cost, and/or have relatively simple data acquisition and processing, and seamless extraction of plant traits, including crop biomass and height. Implementations of the sensor system may be relative light weight. Implementations of the sensor system may provide rapid data collection in the field of the crop, including spatially-located (geolocated) crop height measurements, injection of data onto the remote computing system via a wireless Internet connection, and automated data processing. Implementations of the sensor system described herein may provide better accuracy in phenotyping crop genotypes compared to ultrasonic systems, including due to a higher sampling rate, using of multiple stacked detectors, and/or a focused field of view (FoV). Implementations of the sensor system described herein may produce significantly less voluminous measurement data, allowing for improved communication with a remote computing system for easier cloud uploading and processing. The sensor system may be able to non-destructively estimate plant biomass and height using the integrated ground-based sensor with an end-to-end pipeline in data acquisition through to the IoT-based cloud uploading and processing. Moreover, high temporal resolution data provides the opportunity to study dynamic crop responses to the environment to evaluate genotype performance.

In experimental testing of an implementation of the sensor system described hereinafter, crop fresh biomass, dry biomass and plant height estimated by the sensor system results had high correlations with comparison measurements (including ground-truth manual measurements or accurate reference LiDAR imaging measurements) in a wheat field trial and in a ryegrass field trial. In the context of precision agriculture, plant biomass and height are valuable traits for making informed management decisions, and the proximal sensor system is able to estimate these without damaging the in-season crop. The sensor system can be readily mounted on a tractor or boom-spray to collect field measurements. The adopted agronomic design of the small-scale field experiment enables direct transferability of the established biomass and height estimation models to a conventionally managed larger-scale farmer's field. Furthermore, the presented method of modelling biomass in wheat and ryegrass could be suitably extended for non-destructive in-season estimation of biomass in other field crops including vegetable, grain and forage crops.

### System and Method

As shown in FIG. 1, described herein is a measurement system 100 including:
a. a sensor system 300;
b. a mobile/vehicle mount 102 configured to hold/support the sensor system 300 above a crop 104 (of field/pasture plants, e.g., a plot); and
c. a remote computing system 106, e.g., which can include a cloud-computing server accessed via the Internet 108, configured to communicate with the sensor system 300 using at least one data communication protocol and connection, e.g., a wireless connection/link 110 (which can include a radio-frequency carrier) and a wireless network 112 (providing a wireless Internet connection, e.g., via a cellular data network and/or via local area network (LAN)), and including a master data repository configured to store the measurement and geolocation data.

As shown in FIG. 2 and described hereinafter, the measurement system 100 is configured to perform/execute a measurement method/process 200 (“method 200”) which includes:
a. the sensor system 300 automatically measuring heights of the crop 104 while being held/supported by the mount 102 moving over/across the crop 104 (202);
b. the sensor system 300 automatically and wirelessly sending data representing the corresponding measured heights to the remote computing system 106 (206); and
c. the remote computing system 106 determining/calculating/estimating phenotypic quantities (“phenotypic measurements”) of the crop 104 based on the data representing the corresponding measured heights and geolocations (208) for the purpose of high-throughput phenotyping.

As shown in FIG. 2 and described hereinafter, the measurement method 200 includes:
a. the sensor system 300 automatically measuring/determining a geolocation of each height measurement by simultaneously measuring the geolocation of the sensor system 300 while measuring the heights (204);
b. the sensor system 300 automatically and wirelessly sending data representing the corresponding measured geolocations to the remote computing system 106 (206); and
c. outputting the phenotypic measurements (210) to machine-readable memory (e.g., the master data repository) and/or to a user device for display to a user (e.g., a farmer or scientist)

### Sensor System

As shown in FIG. 3, the sensor system 300 includes:
a. a LiDAR module 302, configured to make range measurements, thus generating measurement data representing the range measurements, to measure the heights of the crop 104—the LiDAR module 302 includes a LiDAR sensor 303;
b. a computing module 304, including a microprocessor 322, machine-readable memory readable/writable by the microprocessor 322 (e.g., a Raspberry Pi (TM) 4 computer), and at least one wireless communications module 326 (e.g., including a microchip and/or antenna) configured for the computing module 304 to communicate using the wireless connection 110 (wherein the wireless communications module 326 and its antenna are configured to communicate according to a wireless data protocol, e.g., a cellular protocol—including an Internet-of-Things (IoT) protocol—defined by the ITU (e.g., LTE, 2G/3G/4G/5G/6G, NB IoT), a wireless local area network (WLAN) protocol (e.g., WiFi, 2.4 GHz and 5.0 GHz IEEE 802.11ac), and/or a Bluetooth protocol (e.g., 5.0, BLE)); and
c. a GNSS module 306 with a GNSS receiver 308 configured to simultaneously measure the geolocation of the sensor system 200 while the LiDAR module 302 is measuring the heights (e.g., based on a GNSS logger, e.g., based on a Navio (TM) unit from Emlid Ltd., Hong Kong).

By way of the LiDAR module 302, the sensor system 300 is configured for non-contact/remote sensing/measurement of the crop 104, thus mitigating/avoid damage to the crop 104 during the measurements, allowing for repeated/continuous measurements without damaging the crop 104.

As shown in FIG. 3, the sensor system 300 may also include a power source 310. The power source 310 may include a battery 312, e.g., a 12-volt battery, that powers the LiDAR module 302. The power source 310 may include a DC-to-DC converter 314, powered by the battery 312, that provides an output voltage connector 316 with a different voltage from that powering the LiDAR module 302, e.g., 5 volts, to power the computing module 304 and the GNSS module 306 with the GNSS receiver 308.

The LiDAR module 302 is configured to make the range measurements (also referred to as “LiDAR measurements”) substantially downwards from the LiDAR module 302 to the crop 104 due to the mounting/positioning/orientation of the LiDAR module 302 on the mount 102. The range measurements are indicative of the crop height measurements as described hereinafter. The LiDAR module 302 is configured to specifically measure range in the selected direction (downwards) within a required/predefined Field of View (FoV) of the LiDAR sensor 303.

The LiDAR sensor 303 is configured for one-dimensional scanning (which is across-track scanning when in use), which can be only one-dimensional (1D) scanning along the across-track direction (referred to as “a first horizontal direction” or “horizontal scanning direction”) because scanning along the back-to-front direction (referred to as “a second horizontal direction” or “horizontal travel direction”, which is at least partially, and/or substantially (which is typical), perpendicular to the first horizontal direction) is provided by movement of the mount 102. This raster-like scanning along the two mutually perpendicular horizontal directions generates the two-dimensional images. The ID scanning LiDAR sensor 303 (i.e., configured for 1D scanning) includes a solid-state LiDAR sensor. The solid-state LiDAR sensor may include a micro-electromechanical system (MEMS) chip or an optical phased array to steer a laser beam from the LiDAR sensor 303 along the first horizontal direction. By including solid-state components of the LiDAR sensor, which can be the MEMS chip or phased array, to steer the laser beam, the solid-state LiDAR sensor may thus have no mechanical moving parts larger than elements of the MEMS chip, e.g., the solid-state LiDAR sensor may thus have no mechanical moving parts larger than 0.1 mm average diameter. The LiDAR sensor 303 may have a relatively narrow across-track scanning distance (along the first horizontal direction) to scan only the crop's canopy profile. The across-track scanning distance may correspond to an across-track FoV of less than 90 degrees, less than 60 degrees, less than 50 degrees, between 35 degrees and 60 degrees, or between 45 degrees and 50 degrees, e.g., substantially 48 degrees. By using 1D scanning and the relatively narrow across-track scanning distance, the LiDAR sensor 303 may generate significantly less data output and processing overload compared to other LiDAR units, e.g., 360-degree LiDAR scanners. Using the 1D scanning may allow the LiDAR sensor 303 to be smaller, lighter and/or simpler that other LiDAR units, e.g., 360-degree LiDAR scanners, and/or may allow it be attached easily to any mount or vehicle, e.g., existing farm equipment/vehicles (e.g., a watering boom or a fertilizer boom), e.g., because it is small and not heavy (and can be moved/attached manually) and/or because it draws less power (and generates less heat) than an 3D imaging system. The LiDAR module 302 is configured to send pulsed light in the laser beam (from the light/laser emitter 318) down to the crop 104, and to detect (in a light receiver 320) the pulsed light reflected from the crop 104 within the FoV. The LiDAR module 302 is configured to measure the reflected pulses in a plurality of discrete beam segments 1102 (e.g., 4 to 16, e.g., 8) as shown in FIG. 11, wherein the discrete beam segments 1102 are arranged across the across-track scanning distance (and along the first horizontal direction), with each segment 1102 having a fractional FoV of the across-track FoV, which can be a substantially equal fraction each (e.g., 6 degrees each), and each segment 1102 corresponds to LiDAR detector element in the LiDAR sensor 303. The LiDAR sensor 303 may have an along-track FoV that is substantially perpendicular to the across-track FoV and that is substantially less than the across-track FoV, e.g., the along-track FoV may be of less than 1 degree, e.g., 0.3 degrees. The along-track FoV is substantially parallel to a direction of travel of the mount 102 through/along/over the crop 104, and the across-track FoV is thus substantially across the plot/portion of the crop 104 under the mount 102. The LiDAR module 302 may have an across-track FoV of Φ=substantially 48° in the across-track (or “side-to-side”) direction. The plurality of LiDAR detector elements, e.g., 8, may be mutually stacked. Each of the stacked detector elements may have a mutually equal horizontal FoV, e.g., θ=6 degrees, covering the plots along the widthwise direction, AB, as shown in FIGS. 11 and 12. As mentioned hereinbefore, the LiDAR sensor 303 may thus be described as configured for 1D scanning (in the across-track direction) because scanning in the along-track direction is provided by movement of the mount 102. The LiDAR module 302 may be based on a sensor from Leddar Tech (TM), Quebec, Canada.

The LiDAR sensor 303 includes the laser emitter 318 which may be configured to operate over at least one near-infrared (NIR) wavelength (e.g., between 700 nm and 3,000 nm, or between 700 nm and 2,500 nm, or between 700 nm and 1,400 nm, or substantially 905 nm). The laser emitter 318 may have at least a Class 1 eye safety rating, e.g., according to IEC 60825-1:2014. The laser emitter 318 and the corresponding light receiver 320 are powered by the power source 310, e.g., by the battery 312 at a voltage supplied by the battery 312 (not requiring the DC-DC converter 314) which may be 12V±0.6 DC. The laser emitter 318 and the light receiver 320 of the LiDAR module 302 are mounted/directed substantially downward to face the crop 104 under the mount 102 to direct the laser emitter 318 towards the crop 104. The laser emitter 318 and light receiver 320 both face down when mounted to the mount 102, and thus the LiDAR module 302 may be referred to as having a nadir orientation (i.e., looking down). The laser emitter 318 and light receiver 320 may be mutually separated as shown in FIG. 3, or may substantially overlap, so long as they have a nadir orientation. The LiDAR module 302 may have a power consumption from the power source 310 of between 0.5 and 10 watts (W), e.g., between 1 and 3 W, e.g., substantially 2 W. The LiDAR module 302 includes a carrier board (printed circuit board) that hosts an electrical and communication interface 323 of the LiDAR module 302, which includes a plurality of communication interfaces, e.g., SPI and/or USB-CAN-serial (UART/RS-485). The LiDAR module 302 may be configured to have a programmable/selectable data refresh rate, measurement accumulation, and/or a sensitivity peak, and the LiDAR sensor 303 may be tuned based on the type of the target vegetation in the crop 104.

The mount 102 is configured to hold/support the sensor system 300 above a crop 104, and to direct the beam of the LiDAR module 302 substantially downwards towards the crop 104, thus holding/supporting the sensor system 300 in a location/position/orientation such that it measures the distance between its LiDAR module 302 (on the mount 102) and the crop 104, at least a top layer/canopy of the crop 104. As described hereinafter, the measurement system 100 is configured to measure the height of the crop 104 based on a difference between the height of the LiDAR module 302, which is referred to as its “mounted height” (i.e., the selected height of LiDAR module 302, and thus the LiDAR sensor 303, above the ground/soil, marked “D” in FIG. 12) and the measured distance between the LiDAR module 302 and the crop canopy. The laser emitter 318 and the light receiver 320 are oriented substantially downward such that the crop 104 substantially fills the FoV. The mobile/vehicle mount 102 (or “field mount” or “rover”) may include wheels configured to roll the sensor system 300 along the ground/soil (e.g., substantially parallel to the ground) under the crop 104. The mount 102 with the wheels may include a manual push-type vehicle or a motor, thus forming a motorised vehicle/mount, which may include a tractor or a spray boom, a watering boom or a fertilizer boom. The mount 102, its motor (if present), its control/steering system, and its wheels are configured to move the sensor system in the selected horizontal travel direction (e.g., along a lengthwise direction 802 shown in FIG. 8) of the mount that is at least partially transverse to the across-track horizontal scanning direction (the first horizontal direction) of the laser emitter 318. The mount 102 may include a ground vehicle/mount with the wheels, and may have a wheelbase or width of at least 1.25 m to enable traversing the sensor system 300 along the lengthwise direction 802 of the crop as shown in FIG. 8. The mounted height may be between 1 m and 10 m, or between 1.2 m and 3 m, or substantially 1.8 m. Having the mobile/vehicle mount 102 in the form of the ground vehicle/mount with wheels may be preferable in some applications, e.g., where aerial vehicles cannot operate with sufficient stability or for sufficient durations. In implementations, the mobile/vehicle mount 102 may include the wheeled vehicle/mount in the form of a side-by-side vehicle (“SxS” or “SSV”), an unmanned ground vehicle (which has a space below the vehicle, as used agriculture research fields), or a mower (configured to mow the field crop). The mobile/vehicle mount 102 may include a mounting system or attachment system that is configured to hold/support the sensor system 300 onto the wheeled vehicle/mount, e.g., including at least one bracket and at least one fastener, including manually operable brackets/fasteners such that the sensor system 300 can be manually attached to the wheeled vehicle/mount in its held/supported location/position/orientation, allowing the sensor system 300 to measure the crop heights; and such that the sensor system 300 can be manually detached/removed from the wheeled vehicle/mount after the measuring—being able to simply attach and operate the sensor system 300 demonstrates its modularity and ease of use.

The computing module 304 is configured to provide a sensor driver unit. The computing module 304 may include single-board computer (SBC), e.g., a Raspberry Pi 4 4 GB Model B. The computing module 304 may have a compact size, e.g., as small as (or smaller than) the size of a credit card (e.g., a width and a height each less than 150 mm, and a depth less than 15 mm). The microprocessor 322 (“onboard microprocessor”) may provide relatively decent processing power, e.g., at least substantially equivalent to a 1.5 GHz quad-core Cortex-A72 (ARM v8) 64-bit System-on-Chip (SoC). The memory may include at least 4 GB of onboard memory 324, including synchronous dynamic random-access memory (SDRAM) storage (e.g., LPDDR4-3200). The computing module 304 may include wireline/wired communications modules configured for the computing module 304 to communicate with the LiDAR module 302 and the GNSS module 306 to acquire/receive the measurement data and the geolocation data respectively, e.g., via USB with the wireline/wired communications modules including a USB module and USB port (e.g., including USB 3.0 ports, and USB 2.0 ports), and/or via a (40-pin) general-purpose input/output (GPIO) header/port with the wireline/wired communications modules including a GPIO module and GPIO port. The memory may include removable memory 328 for loading an operating system and data storage, e.g., a Secure Digital card and an SD card slot (e.g., micro-SD). The operating system may be a Linux-based operating system, e.g., Raspbian Buster (TM). The memory includes an onboard data storage system. The memory includes one or more operational modules configured to be executed by the operating system, and configured to: (i) acquire the geolocation and measurement data from the GNSS module 306 and the LiDAR module 302, (ii) optionally process the acquired data onboard the computing module 304 to generate processed measurement/geolocation data respectively, and (iii) upload the acquired and/or processed measurement/geolocation data to the remote computing system 106, which can include a cloud-computing server access via the Internet 108. The operational modules may be compiled from source files written in C++ and/or Python. For a 32 GB internal memory card, 6 GB may be invested in system files, packages, and the operational modules, leaving about 26 GB storage of the measurement data and geolocation data. For a data rate of about 400 Kbytes/minute, an example onboard data storage system could last for up to approximately 50 days in a continuous mode of operation, without cloud uploading. If the data are more frequently uploaded to the remote computing system 106 in the “cloud”, the onboard data storage space is automatically cleaned up by the computing module 304, thus providing unlimited practical storage. The computing module 304 may be powered by the power source 310, including by the output voltage connector 316, e.g., at 5V DC via a USB-C connector or GPIO header of the computing module 304. The computing module 304 may require relatively low power, e.g., less than 3 Amps at 5 Volts, i.e., less than 15 Watts.

The GNSS module 306 is a form of a global navigation system receiver module configured for geolocating/tagging the LiDAR measurements with their respective geolocations, e.g., as data in positioning logs. The GNSS module 306 may include a GNSS receiver 308 (e.g., from Emlid Ltd., Hong Kong). The GNSS module 306 may be configured to support GPS, GLONASS, Beidou, Galileo, and/or SBAS satellite constellation systems. The GNSS module 306 may be relatively low cost and relatively reliable compared to other commercial-grade positioning sensing systems. As shown in FIG. 3, the GNSS module 306 may include: a (dual) inertial measurement unit (IMU) to improve/correct the geolocation measurements; an RC input/output co-processor 332; a barometer chip 334 to improve/correct the geolocation measurements; and a GNSS receiver chip 336 to communicate with and receive data from the GNSS receiver 308.

Once the sensor system 300 is powered on, e.g., manually, the operating system and the operational modules are configured to automatically connect the sensor system 300 to the wireless network 112 via the predefined wireless connection 110 (e.g., WiFi, etc.) available in the field, to connect with the master data repository of the remote computing system 106 that is configured to store the range and geolocation data. The memory may include credentials (including a password and/or a subscriber identity module (SIM)) which the operational modules use to automatically connect to the wireless network 112, e.g., a password-protected hotspot or cellular network. Alternatively, in the absence of a nearby/available wireless network 112, the sensor system 300 enters in a non-networked mode in which the acquired measurement and geolocation data are saved onto the computing module's memory and uploaded to the remote computing system 106 once the wireless connection 110 has been established, e.g., by later logging onto the password-protected hotspot. Once the acquired data have been uploaded to the remote computing system 106, the operational modules are configured to compress a local copy of the acquired data in an archive in the master data repository for safe-keeping, while older data points are automatically deleted as the memory of the computing module fills to free up system space.

Once the sensor system 300 is powered ON, the LiDAR module 302 and the GNSS module 306 may require less than 60 seconds, e.g., around 20 seconds, to initialize, load their required packages, and establish their respective data connections with the computing module 304.

The sensor system 300 can include an indicator (which can be a visual indicator, e.g., an LED, mounted to/on an enclosure of the sensor system 300), driven by the computing module 304 (e.g., connected to a physical IO pin of the computing module 304), in which one of the operational modules is configured to indicate a status of the sensor system 300 to the user. The status (or “state”) of the sensor system 300 is recorded and updated in the memory by the operational modules. The status can include: INITIALIZATION, after the sensor system 300 has been powered on, but before it is ready to make the measurements (during which the indicator can show a steady solid signal, e.g., driven by a HIGH signal on the connected IO pin); READY (or “paused”), after the initialization, when the LiDAR module 302 and the GNSS module 306 are ready to commence data acquisition but have not commenced (during which the indicator can flash rapidly, e.g., at a frequency of around 20 Hz, e.g., driven by a modulated signal on the connected IO pin); and ACQUISITION (or “active”), after the READY state, during which the LiDAR module 302 and the GNSS module 306 acquire the height and geolocation data (during which the indicator can show a steady solid signal, e.g., driven by a HIGH signal on the connected IO pin). The sensor system 300 is configured to transition between the READY and ACQUISITION states multiple times. The sensor system 300 can include at least one manual control element, including a switch and/or button (e.g., active-low with internal pull-up resistance), that, when manually activated, generating signals (“trigger signals”) for the sensor system 300 to transition between the states. Critical settings for the GNSS module 306 and the LiDAR module 302, e.g., sampling frequency, signal strength, accumulation rate, and time tag format, are predefined as default parameters for ease of in-field operation.

The power source 310 may include voltage regulator circuitry that is configured to provide a steady and/or filtered DC output power, e.g., a 12 V constant output, for the LiDAR module 302. The power source 310 may provide a step-down 5 V output to power the computing module 304 and the GNSS module 306 in the form of the DC-DC converter 314. The power source 310 may provide total current consumption rated between 500 mA and 1,000 mA, e.g., around 800 mA, in the regular mode of operation to power the LiDAR 302, the computing module 304 and the GNSS module 306. The sensor system 300 may be configured to operate for up to 3 hours with the battery 312 in the form of a portable 2500 mAh battery. The battery 312 can be swapped manually to extend the in-field operational time.

As shown in FIG. 3, the LiDAR module 302 is communicatively connected to the computing module 304 by a standard-defined interface, e.g., using a USB port on the computing module 304 and USB-CAN-serial communication. The GNSS module 306 (with the GNSS receiver 308) is communicatively connected to the computing module 304 by a standard-defined interface, e.g., via a GPIO header/port of the computing module 304. Inclusion of the standard-defined interfaces in the sensor system 300 allows easy replacement of any one of the three modules 302,304,306 if required, e.g., due to damage during in-field use.

### Sensor Case

As shown in FIG. 4, the sensor system 300 includes at least one sensor case 500 that is configured to surround, enclose and encase electronic circuitry portions of the LiDAR module 302, the computing module 304 and the GNSS module 306—thus the LiDAR module 302, the computing module 304 and the GNSS module 306 may be described as “integrated” together in the case 500. The sensor case 500 may include the power source 310, or alternatively, the power source 310 may include its own power case/housing that surrounds, encloses and encases electronic circuitry portions power source 310. The sensor case 500 and the power case surround and seal off the enclosed circuitry portions to mitigate/stop the ingress of moisture/dust/dirt while the sensor system 300/power source 310 is operating in the field. As shown in FIG. 4, portions of the LiDAR module 302 may extend from the sensor case 500, e.g., the light receiver 320, and/or an electrical connection to the power source 310. The manual control element and the indicator may be mounted on/to the sensor case 500 to provide convenient manual access, e.g., on a top or side of the case 500. The sensor case 500 (or “enclosure”) may be formed/manufactured of an additive/3D printing material, using an additive/three-dimensional (3D) printer (e.g., from Geldermalsen, The Netherlands). The additive/3D printing material may include a polymer material, which can be polymer filament, e.g., acrylonitrile butadiene styrene (ABS) plastic filament (from Geldermalsen, The Netherlands). As shown in FIG. 5, the sensor case 500 may include a plurality of portions/parts/pieces/sides, substantially forming a rectangular prism, each formed/manufactured of the additive/3D printing material, including: a left piece 502, a back piece 504, a right piece 506, a bottom piece 508, a front piece 510 and a top piece 512. Each of the plurality of pieces may be manufactured/printed separately. The plurality of pieces may be 3D printed, together, lying flat on the build plate, which may allow for stronger cross-sectional adhesion between the layered threads of the filament compared to printing the portions vertically, producing stronger printed pieces. After manufacture/printing, the plurality of pieces may be mutually assembled/fastened using threaded fasteners (screws or bolts), e.g., M3 bolts. The sensor case 500 may include compressible/deformable seals/gaskets between mutually assembled ones of the portions, e.g., polymer rings, or “O” rings. The primary parameters for the 3D printer configured to manufacture/print the pieces may include one or more of: Layer Height of substantially 0.1 m; Wall thickness of substantially 1.2 m; Infill Density of substantially 100%; Infill Pattern of substantially Cubic; Printing Temperature of substantially 245° C.; Build Plate Temperature of substantially 85° C.; Print Speed of substantially 25 mm/s; and Cooling Fan speed of substantially 20%.

The sensor system 300 with the LiDAR module 302, the computing module 304, the GNSS module 306 and the sensor case 500 may have a weight of less than 1 kilograms (kg), or less than 550 grams (g), which is relatively light-weight compared to other digital sensing technologies. The LiDAR module 302 may have a weight of less than 200 g, the computing module 304 may have a weight of less than 50 g, the GNSS module 306 (with the GNSS receiver 308) may have a weight of less than 100 g, and/or the sensor case 500 may have a weight of less than 200 g. In implementations, the total weight of the sensor system 300 (including the LiDAR module 302, the computing module 304, the GNSS module 306 and the sensor case 500, but not the power source 310) was within the range 350 to 500 g, e.g., approximately 400 g. Approximate weights of example implemented components described herein were as follows: the computing module 304 with the Raspberry Pi (TM) was 46 g, the Navio (TM) unit was 23 g, the Leddar Tech (TM) LiDAR module was 144 g, the 3D print enclosure was 118 g, and other elements of an example sensor system (including wire, the GNSS receiver, the LED switch) were 67 g; thus the total weight of the example sensor system with the power source 310 was 398 g (or substantially 400 g).

The mount 102 is configured to hold/support the sensor system 300 (including the power source 310) on itself, e.g., by way of fasteners (such as straps/clips) and a platform (e.g., a mesh), as shown in FIGS. 8 and 9. The mount 102 is configured to the sensor case 500 and the power case/housing mutually adjacent on the platform, e.g., as shown in FIGS. 8 and 9, such that: (i) the laser emitter 318 is directed towards the crop 104, and (ii) the power source 310 is electrically connectable to, or connected to when in use/operation, the LiDAR module 302 and the computing module 304.

### Remote Computing System

The remote computing system 106 (which may be referred to as a “remote server”) includes machine-readable memory (“server memory”) and one or more microprocessors (“server microprocessors”) connected to perform operations by executing server operational modules (“server modules”) in the server memory. The server modules may include data processing modules referred to as “high-level processing nodes” that are configured to control the server microprocessors to provide high-level functions on the data send from the sensor system 300. The server modules may be configured to execute automatically and immediately when new acquired data is sent from the sensor system 300 to the remote computing system 106. These high-level processing nodes may be provided in the remote computing system 106 instead of the computing module 304 because the server microprocessors may have substantially more processing power than the onboard microprocessor 322 and/or to mitigate power drain and overheating of the onboard microprocessor 322 and memory. The high-level processing nodes (of the server modules) may be configured to automatically analyse/process the measurement and geolocation data on receipt. The high-level processing nodes may be based on source code, e.g., written in Python 3.7.8, and may use available source packages, including os, fnmatch, matplotlib, numpy, skimage, and opencv2. The high-level processing nodes may include: a range-to-height conversion module; a denoising module; a segmentation module; a speed-compensation module; an edge-compensation module; a geolocation module; a phenotypic module, which can include a biovolume module; and an output module.

### Calibration

The measurement system 100 may include a calibration apparatus 600, and the onboard memory may include calibration data generated from (i.e., empirical calibration) of the LiDAR module 302 using the calibration apparatus 600. As shown in FIG. 6, the calibration apparatus 600 includes: a plurality of legs (e.g., a tripod 602) for holding the sensor system 300 above a hard, flat area/surface 606 (which provides a calibration area/reflector bigger than the minimum FoV of the LiDAR module 302); an extensible portion (e.g., an arm 604) configured to adjust a “true” distance of the sensor system 300 from the flat area/surface 606; and an coupling/mount 608 to hold the hold the sensor system 300 on/to the calibration apparatus 600 with its laser emitted oriented towards the area/surface 606. The measurement method 200 includes a calibration process in which a plurality of true calibration distances/heights (of a calibration point on the sensor system 300 above the area/surface 606) are selected using the calibration apparatus 600 (e.g., manually or automatically with a motorised calibration apparatus 600), and optical calibration measurements (from the LiDAR module 302) are recorded and combined with the respective true calibration distances/heights to generate/record a calibration model that relates the LiDAR measurements (inputs) to the true distances/heights (output). The calibration process may include making the LiDAR measurements, e.g., for at least 5 seconds at each distance, and/or using at least nine (9) calibration heights specified, e.g., ranging from 40 cm to 265 cm in steps of approximately 30 cm. The calibration process may be performed for the plurality of detectors in the LiDAR module 302, e.g., 9 calibration points measured for each detector, e.g., for 8 detectors there may be 72 readings forming the calibration model. The calibration model is stored as calibration data in the server memory, and the server modules include a calibration module that controls the server microprocessor to access the calibration model to determine calibrated range measurements (also referred to as “corrected” or “tuned” range measurements) from the LiDAR distance measurements (also referred to as the “raw” LiDAR measurements), and thus to provide precise distance profiles automatically, as described hereinafter.

### Data Acquisition

During the data acquisition stage/state (which may be referred to as a “scanning mission”), including the simultaneous measurement processes 202 and 204 of the measurement method, the sensor system 300 is moved by the mount 102 in a pattern 702 transverse to the crop, e.g., as shown in FIG. 7. The pattern 702 may include a sinuous traverse path/trajectory, alternating in direction between mutually adjacent parallel part portions, to cover each row in an individual range, followed by the next range in a reverse direction. This pattern 702 may enable least movement of the mount 102 or the equivalent time in data acquisition.

The LiDAR module 302 and the computing module 304 may be configured to record the LiDAR measurement data (“data scan”) continuously throughout the “measure crop height” process 202 at a preselected scan rate, e.g., 10 Hz to 120 Hz, e.g., 50 Hz to 70 Hz, e.g., substantially 60 Hz. The LiDAR module 302 may be configured to record the raw range measurements (“r”) for each LiDAR detector, e.g., in a CSV format, and the operational modules of the computing module 304 are configured to control the computing module 304 to send the raw range measurements (“r”) to the remote computing system 106 as soon as possible after the scans, as described hereinbefore.

The calibration module controls the server microprocessor to access the calibration model to determine calibrated range measurements (also referred to as “corrected” or “tuned” height/range measurements) from the raw LiDAR distance measurements and the stored calibration model.

The range-to-height conversion module is configured to control the server microprocessors to convert the calibrated range measurements (r) into crop height measurements (h) using a conversion process represented by trigonometric calculations set out in Equations (1) to (4) hereinafter, where, as shown in FIG. 12, D is the mounting height of the LiDAR detectors above the ground (which is defined by the mount 102 and position of the sensor system 300 thereon), r is the raw range measurements collected for a detector, α and β are the orientation angles for a detector element from the vertical axis (SH) to the detector's FoV edge and center respectively (e.g., predefined/measured during the calibration process), θ is the predefined width of each detector element, Φ is the total FoV, n is the number of detector elements, and w is the width traced by a detector with a FoV of θ at an angle of β from vertical axis on the height (h):

$$h = D - r \cdot cos ( β ) \qquad ( 1 )$$

$$β = ( θ / 2 ) + α \qquad ( 2 )$$

$$θ = Φ / n \qquad ( 3 )$$

$$w = r \cdot cos β \{ tan α - tan ( α + θ ) \} \qquad ( 4 )$$

In the remote computing system, D, θ and Φ are stored as parameters in the server memory, whereas the variables r, α, β, h, and w are received/generated/stored/accessed as respective array vectors R, A, B, H, and W, e.g., the array vector H=[h₁, h₂, . . . hₙ] for height.

The LiDAR scan measurements may be collected continuously over the plots along the transects (e.g., as shown in FIG. 7), in which case the height measurements (h) may include spurious or noisy disturbances during the data acquisition due to undulations in the ground surface. The denoising module is configured to mitigate the spurious or noisy disturbances in the height measurements (h) due to the undulations by performing a denoising process on the calculated crop height measurements (h), including filtering the calculated crop height measurements (h) with a smoothing filter, e.g., using a Savitzky-Golay filter; however, certain disturbances due to these undulations may remain in the form of false peaks 1302 near the edges of the plots, e.g., as shown in FIG. 13A. To remove/reduce these false peaks 1302, first, the denoising module is configured to cause the remote microprocessor to classify ground surface measurements in the height measurements (h) and to mask off ground-surface height in the height measurements (h) using a vertical threshold (e.g., Vₜₕ<5 cm) to remove the undulating ground surface. Second, the remote modules are configured to use a horizontal (Hₜₕ) threshold criterion to remove any remaining false peaks 1302 (also referred to as “continuous peak segments”) under a threshold (Hₜₕ) lengthwise scan size of samples (e.g., Hₜₕ<50 samples). As the scan size or scan profile for the plots (e.g., 5 m long) is generally significantly greater than the size of false peaks 1302, the Hₜₕ provides a simple and effective basis to eliminate any slight undulations in the signal profile. An example of a remoted false peak 1402 I shown in FIG. 14B.

The segmentation module is configured to automatically segment the height measurements (h) into mutually separate plot profiles corresponding to mutually separate plots of the crop 104 along a direction of travel of the mount 102 (along the direction of the scan) into a plurality of extracted plot profiles that can be overlapped as shown in FIG. 15.

The speed-compensation module is configured to automatically compensate for variable speed of movement of the mount 102, and thus the sensor system 300 along a direction of travel of the mount 102 along the pattern 702. Even if the mount 102 traverses at a near-constant speed (e.g., 1.4 m/s), maintaining a uniform speed throughout a long scan duration may impractical in field conditions. A lack of the uniform speed leads to mutually variable/unequal lengths 1502 of the number of measurements made for mutually different plots, e.g., as shown in FIG. 15. As the data are collected at a constant rate from the LiDAR module 302 (e.g., 60 Hz), a greater number of samples is captured when the mount speed is slower, and vice versa when the mount speed is faster. In an example, the number of samples for all the plots may range between 210 and 400, as shown in FIG. 15. To adjust for this discrepancy in sample numbers, the lengthwise acquisition of the samples may be resampled at/to a constant selected rate, e.g., m=100 samples per plot of 5 m in length, as shown in FIG. 16, thus the array vector His acquired m times or the resampling frequency for each plot, forming a m×n matrix, represented by Hₚₗₒₜ in Equation (5).

$${H}_{plot} = {[ \begin{matrix}{h}_{11} & … & {h}_{n 1} \\ ⋮ & ⋱ & ⋮ \\ {h}_{m 1} & … & {h}_{mn}\end{matrix} ]}_{m \times n} \qquad ( 5 )$$

The edge-compensation module is configured to automatically remove or add edges from/to the scans, i.e., from the height measurements (h or Hₚₗₒₜ), corresponding to range measurements from outer detector elements of the LiDAR module 302 by automatically adjusting the height values of these edges. As the detector elements are stacked at different angles (α, β) from the nadir, the widthwise/side-to-side footprint (w) increases progressively away from the nadir and reduces with the height (h) of the crop (per Equation 4), and as shown in FIGS. 11 and 12. For an optimum medium-sized plot, the FoV of the sensor covers the entire width 1802 of the crop plot (e.g., as shown in FIGS. 17 and 18). However, a short-sized plot is farther from the sensor, so the FoV covers beyond the width 2002 of the plot (e.g., as shown in FIGS. 19 and 20), so ones of the beam segments 1102 on the edges 2004 are automatically detected and removed by the edge-compensation module. Inversely, tall-sized plots are closer to the sensor, so the FoV covers less than a full width of the plot (e.g., as shown in FIGS. 21 and 22), thus the edge-compensation module extrapolates to account for the missed plants on the edge of the plots: a spatial resampling in the widthwise direction 2202 (to insert missing heights along the edges 2204) is applied by the remote microprocessor, controlled by the edge-compensation module, to adjust for this mismatch. The dimension of the Hₚₗₒₜ matrix remains unchanged in this step, but the internal values are revised.

The geolocation module is configured to automatically geolocate the height measurements, which may be in the form of the Hₚₗₒₜ matrix, based on the geolocation data/tags from the GNSS module 306. The Hₚₗₒₜ represents a mathematical matrix formulation of the collected height profile for each plot. The segmented Hₚₗₒₜ matrices may be geolocated using the corresponding tags collected through the onboard GNSS module 306. The sensor system 300 uses the GNSS module 306 to synchronise its internal clock with GNSS time. The remote microprocessor receives the GNSS timestamps in the sent data from the sensor system 300, and uses the GNSS timestamps to match the geolocation measurements with the height measurements using timestamps in the raw height data (r). The remote modules are configured to control the remote microprocessor to match these measurements to geolocate/register the segmented plot matrix Hₚₗₒₜ, thus generating a geolocated Hₚₗₒₜ.

The phenotypic module is configured to automatically control the remote microprocessor to calculate/measure/estimate a phenotypic measurement from the height measurements. The phenotypic measurement may include a biovolume measurement, and the phenotypic module include a biovolume module that is configured to automatically control the remote microprocessor to calculate/measure/estimate a crop biovolume (BV) from the height measurements. The geolocated Hₚₗₒₜ for individual plots enables geospatial analysis to summarize average height and other statistical measures such as volume of the crop, termed as BioVolume (BV). The biovolume module is configured to automatically calculate the average height (h_{avg}) of each plot according to Equation (6), where, h_{m,n} is a specific element of the Hₚₗₒₜ matrix at m^{th} row and n^{th} column, d corresponds to the detector element, n is the total number of detectors (e.g., n=8), s represents the sample, and m represents the total number of samples per-plot after resampling (e.g., m=100):

$${h}_{avg} = \frac{1}{m \times n} {\sum }_{s = 1}^{s = m} {\sum }_{d = 1}^{d = n} {h}_{m , n} \qquad ( 6 )$$

The biovolume module is configured to automatically calculate/measure/estimate the BV for each plot according to Equation (7), where, h_{m,n} is a specific element of the Hₚₗₒₜ matrix at m^{th} row and n^{th} column, x and y specifies the width (across-track, or side-to-side) and lengthwise (along-track, or back-to-front) directions of the plots respectively, X and Y represent to the dimensions of the plot, i.e., width and length respectively, w_{m,n} is the unit width traced by an individual detector at height h_{m,n}, and t (e.g., 5/100 m) is the length traced by the detector according to the resampling:

$$BV = {\sum }_{y = 1}^{y = Y} {\sum }_{x = 1}^{x = x} {h}_{m , n} \times {w}_{m , n} \times t \qquad ( 7 )$$

The output module is configured to automatically output the phenotypic measurements (including the crop height and/or biovolume) to machine-readable memory and/or to a user device for display to a user, e.g., the farmer or a scientist for HTPP. The user device is remote from the remote computing system, e.g., at the field, and/or on the mount 102, e.g., in a tractor carrying the sensor system 300.

### Exemplary Test Implementations

Described hereinafter is a test implementation of the measurement system 100 and the measurement method 200 when used for field testing in a wheat field trial containing multiple genotypes, showing that crop fresh and dry biomass, and estimated plant height had high correlations with manually measured data. The field trial comprised 36 wheat genotypes planted in individual plots, as shown in FIG. 7, with a sowing density of approximately 150 plants per m². The list of wheat genotypes is shown in FIGS. 30-32. Each plot was 1 m (5 rows) wide and 5 m long, as shown in FIG. 7. Field observation, including both automatic measurements with the measurement system 100, and ground truth manual measurements, were performed at 100 and 140 days after sowing (DAS) where plants were at first-node and anthesis growth stages, respectively. Crop height was measured from the ground level to the highest point of the plant, with the average of the four random height measurements per plot. Thereafter, each plot was manually harvested separately and weighed soon after harvest to measure fresh weight (FW) biomass and oven-dried at 70° C. for five days to measure dry weight (DW) biomass. The measurement data were captured on the same days before manual plant height and biomass harvest. As shown in FIG. 23, a calibration model was generated from 72 observations collected at a variable step distance of approximately 30 cm between 40 cm and 265 cm, and the test calibration model included a regression line plot between reference and measured distances in the laboratory, shown in FIG. 23, in which the correlation was very high between each of the sensor's detector output range and the target distance. The fitted models for all detectors explained a 98.5% of the variability of the response. The R2 for all models averaged 0.98 with an RMSE of 3.97 cm, which is very practical for the end-use case of the sensor in phenotyping plant canopies with fragile structures. The average error in absolute value was 3.54 cm. The crop height, FW and DW regression models were validated with the manual data collected at 100 and 140 DAS. The crop height of wheat genotypes in the experiment ranged from 42 to 92 cm on 100 DAS and 64 to 102 cm on 140 DAS with a normal distribution. The mean crop heights were 80 and 84.6 cm on 100 DAS and 140 DAS, respectively. A correlation-based assessment was used to evaluate the sensor-derived height readings (h_{avg}) performance with respect to manual plot height measurements, as shown in FIG. 24. The assessment in FIG. 24 showed a strong linear relationship between sensor height readings and manual crop height with a coefficient of determination (R²) of 0.79, RMSE of 6.09 cm, and MAE of 5.03 cm. Unlike the highest points measured during ground-based surveys, the sensor height readings represent the complete relief of the crop surface; therefore, it may be found to be about 17.5 cm lower than the actual average canopy height. A linear regression model was applied to estimate FW and DW from BV, producing R² of 0.70 and 0.84, RMSE of 490 gm and 138 gm, and MAE of 375 gm and 111 gm, respectively, as shown in FIGS. 25 and 26. A frequency-domain analysis of the residual absolute error showed that the 50% of observations have an absolute error≤4.28 cm for crop height, ≤0.28 Kg for FW and ≤0.09 Kg for DW, when compared with the regression line established by the fitted linear model, as shown in FIGS. 27, 28 and 29. The accuracy in measuring BV in field conditions may influence the final estimated biomass (FW and DW): for example, when estimating canopy BV with a LiDAR sensor, a few centimetres may have some relative effect on the estimation of FW and DW. Nevertheless, the stacked LiDAR unit used here had benefit for better capturing the canopy's spatial profile, which is otherwise challenging with one ultrasonic sensor. The biomass gain between the two time points is also visible in the plots in FIGS. 25 and 26. The fitted linear correlation lines in FIGS. 24, 25 and 26 follow through the sample distribution at each time point, demonstrating the applicability of the methods in progressively measuring biomass estimates. An objective in plant breeding research is to screen genotypes to select better-performing lines with higher growth and yield potential. Non-destructively collected crop height and biomass estimates are crucial contributors in effectively mapping the growth profile. Crop height, FW, and DW estimated across the two time points showed growth trends for wheat genotypes, as shown in FIGS. 30 to 32. The genotypes Aus482 and Aus79 achieved the maximum height of 102 cm amongst all other genotypes on second time point, 140 DAS, as shown in FIG. 30. In terms of biomass, the genotype Aus7992 had the highest FW and DW of 4.5 Kg and 2.1 Kg, respectively, amongst all other genotypes on 140 DAS, and the genotype Cara was shortest and produced minimum FW and DW of 1.1 kg and 1.0 Kg on 140 DAS, as shown in FIGS. 31 and 32. The phenotypic growth profiles measured using the sensor may aid in the genotypic screening of wheat varieties.

In a further test implementation (also referred to as a “use case”), the measurement system 100 and the measurement method 200 were used for field testing in a ryegrass field trial of multiple plots of ryegrass, and height measurements of the ryegrass from the measurement system 100 and the measurement method 200 were shown to be substantially equal or equivalent to comparison height measurements from a commercially available high-resolution 2D distance scanner in the form of an LMS400 sensor from SICK AG (Germany). As shown in FIGS. 33 to 36, the height measurements from the measurement system 100 (shown as dashed lines, labelled “UGV-DBM-Plot”) correlate closely to the comparison height measurements from the LMS400 (shown as unbroken lines, labelled “LMSPlot”) within error/mismatch bounds expected in the art (e.g., due to plant movement in the wind, and registration errors). The further test implementation demonstrated that the measurement system 100 could provide measurements of comparable accuracy to a high-resolution scanner that gathers more data, is larger, requires higher power, more cooling, and/or more complicated/expensive componentry.

### Interpretation

Many modifications will be apparent to those skilled in the art without departing from the scope of the present invention.

The presence of “/” in a FIG. or text herein is understood to mean “and/or” unless otherwise indicated, i.e., “A/B” is understood to mean “A, or B, or both A and B”. The recitation of a particular numerical value or value range herein is understood to include or be a recitation of an approximate numerical value or value range, for instance, within +/−20%, +/−15%, +/−10%, +/−5%, +/−2.5%, +/−2%, +/−1%, +/−0.5%, or +/−0%. The terms “substantially” and “essentially all” can indicate a percentage greater than or equal to 90%, for instance, 92.5%, 95%, 97.5%, 99%, or 100%.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A measurement system including:
a. a sensor system that includes:
i. a Light Detection And Ranging (LiDAR) module with a laser emitter configured to generate measurement data representing raw range measurements to measure heights of a crop, and
ii. a computing module, including:
at least one wireline/wired communications module configured to communicate with the LiDAR module for the computing module to acquire the measurement data from the LiDAR module; and
at least one wireless communications module configured for the computing module to communicate using a wireless connection/link with a remote computing system that is configured receive the acquired measurement data and to determine/calculate/estimate phenotypic quantities of the crop based on the measured heights for the purpose of high-throughput plant phenotyping (HTPP); and
b. a mobile/vehicle mount configured to hold/support the sensor system above the crop and to direct the laser emitter towards the crop.

2. The measurement system of claim 1, wherein the LiDAR module includes a LiDAR sensor configured for one-dimensional (1D) scanning, optionally in a horizontal scanning direction that is at least partially or substantially perpendicular to a horizontal travel direction of the mobile/vehicle mount.

3. The measurement system of claim 2, wherein the LiDAR sensor includes a solid-state LiDAR sensor, optionally including a micro-electromechanical system (MEMS) chip or an optical phased array, configured to steer a laser beam from the laser emitter along the horizontal scanning direction.

4. The measurement system of claim 2, wherein the 1D scanning is over a horizontal scanning distance that corresponds to an across-track field of view (FoV) of the LiDAR sensor, optionally wherein the FoV is less than 90 degrees, or less than 60 degrees, optionally wherein the LiDAR sensor has an along-track FoV that is substantially perpendicular to the across-track FoV and that is substantially less than the across-track FoV, optionally wherein the along-track FoV is less than 1 degree or substantially 0.3 degrees.

5. The measurement system of claim 1, wherein the crop is a field crop or greenhouse crop.

6. The measurement system of claim 1, wherein the sensor system includes at least one sensor case that is configured to surround, enclose and encase electronic circuitry portions of the LiDAR module and the computing module to seal off the enclosed circuitry portions to mitigate/stop ingress of moisture/dust/dirt while the sensor system is operating in a field, optionally wherein the sensor case includes a plurality of portions formed/manufactured of an additive/3D printing material, optionally wherein the plurality of portions are mutually assembled/fastened by threaded fasteners, optionally wherein the sensor case includes compressible/deformable seals/gaskets between mutually assembled ones of the portions, optionally wherein the mobile/vehicle mount is configured to hold/support the power case/housing such that laser emitter is directed towards the crop.

7. The measurement system of claim 1, wherein the sensor system includes a power source, optionally wherein the power source includes a battery that powers the LiDAR module, optionally wherein the power source includes a DC-to-DC converter powered by the battery that provides a different voltage from that powering the LiDAR module to power the computing module, optionally wherein the power source includes a power case/housing that surrounds, encloses and encases electronic circuitry portions of the power source to seal off the enclosed circuitry portions to mitigate/stop ingress of moisture/dust/dirt while the power source is operating in a field, optionally wherein the mobile/vehicle mount is configured to hold/support the power case/housing such that the power source is electrically connected/connectable to the LiDAR module and the computing module.

8. The measurement system of claim 1, wherein the sensor system includes a global navigation satellite system (GNSS) module with a GNSS receiver configured to simultaneously measure the geolocation of the sensor system while the LiDAR module is measuring the heights, optionally wherein the computing module includes at last one wireline/wired communications module configured to communicate with the GNSS module for the computing module to receive the geolocation data, optionally wherein the sensor system includes at least one sensor case that is configured to surround, enclose and encase electronic circuitry portions of the GNSS module to seal off the enclosed circuitry portions to mitigate/stop ingress of moisture/dust/dirt while the sensor system is operating in a field.

9. The measurement system of claim 1, wherein the sensor system with the LiDAR module, the computing module, and optionally a GNSS module and optionally a sensor case has a weight of less than 1 kilogram (kg), or less than 550 grams (g), or between 350 and 500 g; optionally wherein the LiDAR module has a weight of less than 200 g, the computing module has a weight of less than 50 g, the GNSS module has a weight of less than 100 g, and/or the sensor case has a weight of less than 200 g.

10. The measurement system of claim 1, wherein the computing module includes credentials configured to automatically connect to a wireless network via the wireless connection/link, optionally wherein the wireless connection/link includes a radio-frequency carrier.

11. The measurement system of claim 1, wherein the mount includes a ground vehicle/mount with wheels configured to roll the sensor system along ground/soil under the crop in a travel direction of the mount that is at least partially transverse to a horizontal scanning direction of the laser emitter, optionally wherein the mount is configured to hold/support the LiDAR module at a selected height above the ground/soil while the LiDAR module is measuring the heights.

12. The measurement system of claim 1, includes the remote computing system, optionally wherein the remote computing system includes machine-readable memory and one or more microprocessors connected to perform operations by executing server operational modules that include data processing modules that include any one or more of:
a. a calibration module configured to determine calibrated range measurements from the raw range measurements and a stored calibration model;
b. a range-to-height conversion module configured to control the server microprocessors to convert the raw or calibrated range measurements into crop height measurements;
c. a denoising module configured to mitigate spurious/noisy disturbances in the crop height measurements due to undulations of ground under the crop by performing a denoising process on the crop height measurements, including:
i. filtering the crop height measurements with a smoothing filter,
ii. removing ground-surface heights in the crop height measurements using a vertical threshold to remove undulating ground surface heights, and/or
iii. removing false peaks under a horizontal threshold lengthwise scan size of samples;
d. a segmentation module configured to automatically segment the crop height measurements into a plurality of mutually separate plot profiles corresponding to respective mutually separate plots of the crop along a direction of travel of the mount;
e. a speed-compensation module configured to automatically compensate for variable speed of movement of the sensor system along a direction of travel of the mount by resampling the crop height measurements to a constant selected rate for each of the plurality of separate plot profiles;
f. an edge-compensation module configured to automatically remove or add edges from/to the crop height measurements corresponding to range measurements from outer detector elements of the LiDAR module by automatically adjusting the height values of these edges;
g. a geolocation module configured to automatically geolocate the crop height measurements based on geolocation data/tags from the GNSS module;
h. a phenotypic module configured to automatically control the remote microprocessor to calculate/measure/estimate a phenotypic measurement from the height measurements, optionally wherein the phenotypic measurement includes a biovolume measurement;
i. a master data repository configured to store the range measurements, the crop height measurements, the phenotypic measurements, and/or geolocation data; and
j. an output module configured to automatically output the phenotypic measurements to machine-readable memory and/or to a user device for display to a user.

13. A measurement method/process that includes:
a. a sensor system automatically measuring heights of a crop using Light Detection And Ranging (LiDAR) while being held/supported by a mount moving over/across the crop; and
b. the sensor system automatically wirelessly sending data representing the corresponding measured heights to a remote computing system for high-throughput plant phenotyping (HTPP).

14. The measurement method/process of claim 13, wherein using the LiDAR includes one-dimensional (1D) scanning, optionally including using a solid-state LiDAR sensor, optionally including using a micro-electromechanical system (MEMS) chip or an optical phased array configured to steer a laser beam from the laser emitter along a horizontal scanning direction, optionally wherein the 1D scanning is over a horizontal scanning distance that corresponds to a field of view (FoV) of less than 90 degrees, or less than 60 degrees.

15. The measurement method/process of claim 13 including:
a. the remote computing system automatically determining/calculating/estimating phenotypic quantities (“phenotypic measurements”) of the crop based on the received data representing the corresponding measured heights for the purpose of the HTPP; and
b. the remote computing system automatically outputting the phenotypic measurements to machine-readable memory and/or to a user device for display to a user.

16. The measurement method/process of claim 13 wherein the crop is a field crop or greenhouse crop.
